(19) United States
(12) Patent Application Publication
(10) Pub. No.: US 2019/0099540 A1
(43) Pub. Date: Apr. 4, 2019

(54) A CLOSED SYSTEM FOR LABELLING AND SELECTING LIVE CELLS

(71) Applicant: HITACHI CHEMICAL ADVANCED THERAPEUTICS SOLUTIONS LLC, Allendale, NJ (US)

(72) Inventors: David Smith, Ridgewood, NJ (US); Wai Shun Chan, Hackensack, NJ (US); Brian Hampson, Hawthorne, NJ (US); Robert Preti, Ridgefield, CT (US); Yajuan Jiang, Mahwah, NJ (US); Courtney Leblon, Parsippany, NJ (US)

(73) Assignees: HITACHI CHEMICAL ADVANCED THERAPEUTICS SOLUTIONS, LLC, Allendale, NJ (US); HITACHI CHEMICAL ADVANCED THERAPEUTICS SOLUTIONS, LLC, Allendale, NJ (US)

(21) Appl. No.: 16/083,263

(22) PCT Filed: Mar. 27, 2017
(86) PCT No.: PCT/IB2017/051736

§ 371 Date: Sep. 7, 2018

Related U.S. Application Data
(60) Provisional application No. 62/304,781, filed on Mar. 7, 2016.
(60) Provisional application No. 62/305,779, filed on Mar. 9, 2016.

(51) Int. Cl. A61M 1/36; G01N 33/487; C12N 5/00; C07K 16/28; G01N 33/569; G01N 33/543; A61K 35/14; A61M 1/02

(52) U.S. Cl. CPC A61M 1/362; G01N 33/487; A61M 1/3618; A61M 1/3693; A61M 1/029; C07K 16/28; G01N 33/56966; G01N 33/54313; A61K 35/14; C12N 5/0081

(57) ABSTRACT

The described invention provides an automated, closed system and method for separating/isolating a target cell type from a heterogeneous cell population.

### CROSS REFERENCE TO RELATED APPLICATIONS

This application claims the benefit of priority to U.S. Provisional Application No. 62/304,781 (filed Mar. 7, 2016), entitled “A Closed System for Labelling and Selecting Live Cells,” and to U.S. Provisional Application No. 62/305,779 (filed Mar. 9, 2016), entitled “A Closed System for Labelling and Selecting Live Cells.” The entire content of each application is incorporated by reference herein.

### FIELD OF THE INVENTION

The described invention generally relates to cell labeling, cell separation and the isolation of pure populations of cells from heterogeneous cell suspensions.

### BACKGROUND OF THE INVENTION

### Cell Separation

Cell separation is a powerful tool that is widely used in biological and biomedical research and in clinical therapy. The ability to sort cells into distinct populations enables the study of individual cell types isolated from a heterogeneous starting population (i.e., admixture) without contamination from other cell types (Tomlinson M J et al. J Tissue Eng January-December 2013 vol. 4 2041731412472690). This technology underpins many discoveries in cell biology and is further enabling research in areas as diverse as regenerative medicine, cancer therapy and HIV pathogenesis (Yang J et al. Biophys J 1999; 76: 3307-3314; Chan J W et al. Anal Chem 2008; 80: 2180-2187).

### Cell Separation in Biological/Biomedical Research

In biomedical research, cell purification has been widely used for many different purposes, including tissue engineering and regenerative medicine, cancer therapy, and research on the pathogenesis of infectious diseases (Guo K T et al. Stem Cells 2006; 24: 2220-2231; Takaishi S. et al. Stem Cells 2009; 27: 1006-1020; Terry V H et al. Virology 2009; 388: 294-304). Availability of purified cell populations, for example, facilitates diagnosis of clonality in the absence of cytogenetic/molecular markers, contributes to increase the knowledge about intratumoral cytogenetic heterogeneity and clonal evolution pathways of different neoplastic as well as nontumoral disorders, and helps in the diagnosis and prognostic assessment of patients with neoplastic (e.g. multiple myeloma and mastocytosis) and nonneoplastic immunological disorders (e.g. primary immunodeficiencies), among other diseases (Teodosio C et al. J Allergy Clin Immunol 2013; 132: 1213-1224; Escribano L et al. J Allergy Clin Immunol 2009; 124: 514-521; Lopez-Corral L et al. Clin Cancer Res 2011; 17: 1692-1700; Schmidt-Heiber M et al. Haematologica 2013; 98: 279-287; Fernandez C et al. Leukemia 2013; 27: 2149-2156).

### Therapeutic/Clinical Cell Separation

From a clinical perspective, usage of highly efficient cell purification techniques has significantly contributed to the diagnosis and treatment of multiple human diseases (Will B et al. Best Pract Res Clin Haematol 2010; 23: 391-401; Jamieson C H et al. N Engl J Med 2004; 351: 657-667; Majeti R et al. Proc Natl Acad Sci USA 2009; 106: 3396-3401). Therapeutic or clinical cell separation allows for the introduction of enriched cell populations to a patient with a clinical need for those cells, including, for example, separation of leukocytes by aphaeresis (withdrawal of blood; separation into plasma and cells; reintroduction of cells) and enrichment of hematopoietic stem cells by immuno-magnetic separation (Handgretinger R et al. Bone Marrow Transplant 1998; 21: 987-993; To L B et al. Blood 1997; 89: 2233-2258). It also enables the enumeration of cells within an individual's blood system and can aid repopulation of the immune system, for example, in multiple sclerosis patients who have undergone immune ablation treatment (systematic destruction of a patient's immune competence) (Mancardi G and Saccarci R Lancet Neurol 2008; 7: 626-636).

Most regenerative treatments based on cell separation have been restricted to tissues such as blood and bone marrow (To L B et al. Blood 1997; 89: 2233-2258; Stamm C et al. Lancet 2003; 361: 45-46). However, advances in stem cell therapy, tissue engineering and regenerative medicine have revealed the potential for clinical cell-based therapies using cells derived from a variety of tissues, such as adipose and intestine (Zuk P A et al. Mol Biol Cell 2002; 13: 4279-4295; Lanzoni G et al. Cytotherapy 2009; 11: 1020-1031). The use of highly selective cell separation procedures in clinical cell-based treatments has the potential to improve quality of repair and subsequent clinical outcome. Thus, the use of these methodologies in tissue engineering and regenerative medicine has increased, but has not been restricted to these fields. Indeed, cell sorting is used in other scientific areas such as biochemistry, electrical engineering, physics and materials science (Ackerman S J et al. J Biol Chem 2002; 277: 14859-14868; Howard D et al. Biochem Biophys Res Commun 2002; 299: 208-215; Yang J et al. Biophys J 1999; 76: 3307-3314; Chan J W et al. Anal Chem 2008; 80: 2180-2187).

### Cell Purification Techniques

A large variety of cell separation methods are available which are predominantly based on four major features: (i) cell adherence properties; (ii) density and size; (iii) morphological characteristics; and (iv) antibody-binding immunophenotypic properties (Almeida M et al. Pathobiology 2014; 81: 261-275; Tomlinson M J et al. J Tissue Eng 2013; 4: 2041731412472690).

Cell Purification Based on Cell Adherence Properties

Purification techniques which take advantage of unique adhesion properties of a cell population of interest are rather simple, inexpensive and have been extensively used for the isolation of cells from enzymatically digested, mechanically disaggregated and/or explanted primary tissues (Tomlinson M J et al. J Tissue Eng 2013; 4: 2041731412472690). However, in most instances, these techniques do not provide high purity because the adhesion capacity of the cells of interest are also frequently shared by other adherent cells in the sample. Although significant progress has been made regarding the variety and properties of the adhesion surfaces used (e.g. adherence of cells to polymer-brush-grafted glass beads, cell adhesion on micro-/nanostructured surfaces and ligand-specific (protein, peptide and aptamer) cell adhesion), usage of adhesion-based cell isolation has been restricted to applications which do not require high purity or to applications which require negative selection of a specific cell population (e.g. depletion of monocytes from peripheral blood samples) (Nagase K et al. Macromol Biosci 2012; 12: 333-340; Didar T F et al. Lab Chip 2010; 10: 3043-3053). Since an incubation period (at cell culture conditions) is required until adherent cells can be selected or depleted, these techniques can result in microbial contamination of the selected adherent cells and also can modify the biochemical and molecular properties of the selected adherent cells (Tomlinson M J et al. J Tissue Eng 2013; 4: 2041731412472690).

Cell Purification Based on Cell Density and/or Size

Frequently, the unique density and/or size of cells of interest is used for cell purification. Density-based techniques are now mostly based on the use of centrifugation, although historically sedimentation-based methods have been employed (Miller R G, Phillips R A J Cell Physiol 1969; 73: 191-201). The ability to sort large numbers of cells based on their density, relative to a graduated separation medium (usually sugar based), makes these techniques particularly applicable for separations involving the use of blood, which contains 4×10⁹ to 6.5×10⁹ cells/mL. The most commonly used clinical cell separation method is aphaeresis of whole blood to isolate mononuclear cells for treatment of a variety of conditions, including leukemia (Buckner D et al. Blood 1969; 33: 353-369). However, despite the large-scale use of density-based methods, there are still problems with specificity as the differing densities of different cell populations are, in some instances, not large enough to separate out individual cell types. These problems can be overcome, for example, by performing repeated centrifugations using differing concentrations of centrifugation medium and differing angular velocities. By using these techniques, it is possible to isolate different cell types from a complex mix, including disrupted solid tissues (Liu W et al. Proteomics 2011; 11: 2556-3564). Although technically feasible, this is still challenging to perform with high specificity. As such, centrifugation methods are generally used if specificity is not absolutely necessary, as in aphaeresis, or as a pre-enrichment stage to remove cells like red blood cells and platelets (Tomlinson M J et al. J Tissue Eng 2013; 4: 2041731412472690).

Another widely-used, density-based method, mainly used for the isolation of specific subpopulations of mononuclear cells (MNC) from blood-containing samples, is based on antibody-mediated erythrocyte rosetting. This method relies on a combination of antibody binding and density-based cell purification methods. Briefly, undesired cells are specifically labelled with antibodies that subsequently form complexes with erythrocytes forming immuno-rosettes of higher density than that of the cells of interest. After centrifugation of the sample, the immuno-rosettes containing undesired cells are pelleted with the erythrocytes coexisting in the sample, thus allowing the isolation of the target cells (e.g., MNC) at the interphase after density-gradient centrifugation (Strelkauskas A J et al. Clin Exp Immunol 1975; 22: 62-71). These techniques can also be used for positive selection of erythrocyte-rosetting cells. In such cases, further erythrocyte-lysing procedures are required for final purification of the pelleted cells of interest.

Filtration techniques involve the isolation of target cells based on their unique size-associated features. Because filtration techniques are useful approaches for the removal of debris, dead cells and cell aggregates, particularly during the preparation of single cell suspensions from solid tissues. These techniques are relatively simple methods which are generally employed for cell enrichment as a preparative tool for further cell purification steps (Poynton C H et al. Lancet 1983; 1: 524). Depending on the specific cells and/or cellular components to be isolated, filters with different pore sizes and which are built of distinct materials are used (Autebert J et al. Methods 2012; 57: 297-307; Hosokawa M et al. Anal Chem 2010; 82: 6629-6635; Ji H M et al. Biomed Microdevices 2008; 10: 251-257; Lin H K et al. Clin Cancer Res 2010; 16: 5011-5018). Additionally, these methods can be applied for the isolation of large-size cells (Orfao A and Riuz-Arguelles A Clin Biochem 1996; 29: 5-9). However, filtration techniques are usually associated with poor recovery rates due to significant cell loss during the process of filtration.

Other cell sorting techniques exist that combine both cell size and density features. One such technique, centrifugal elutriation, has been successfully used for cell purification purposes. It allows a high recovery of viable cells with relatively low cross-contamination by unwanted cells in a single-step procedure (Bauer K D et al. Cancer Res 1982; 42: 72-78; Chavez-Crooker P et al. J Exp Biol 2001; 2014: 1433-1444; Worthington R E and Nakeff A Blood 1981; 58: 175-178; Schwarze P E et al. Cancer Res 1986; 46: 4732-4737). Cells are targeted through their unique rate of sedimentation; separation is dependent on cell size, the difference between the densities of the distinct cells in the sample, and the selected cell isolation medium (Lindahl P E Nature 1948; 161: 648).

Disadvantages of centrifugal elutriation include the relatively large volume (>100 mL) of various fractions (especially if small numbers of cells are to be separated), the absence of separation using specific features (e.g., surface proteins, cell shape, etc.) and the inability to separate cells which have similar sedimentation properties cannot be separated (See, e.g., Figdor C G et al. J Immunol Methods 1984 Mar. 30; 68(1-2): 73-87).

Cell Purification Based on Antibody Binding

The term “antibody-binding methods” generally refers to the commonly used techniques of fluorescence-activated cell sorting (FACS) and magnetic-activated cell sorting (MACS) (Bonner W A et al. Rev Sci Instrum 1972: 43: 404-409; Miltenyi S et al. Cytometry 1990; 11: 231-238; Rembaum A et al. J Immunol Methods 1982; 52: 341-351). Both technologies utilize cell surface antigens against which antibodies are raised for separation. FACS separation relies on the conjugation of fluorescent labels to these antibodies, whereas MACS uses conjugation to iron oxide containing microbeads. Following binding of conjugated antibodies, FACS and MACS proceed down different routes. FACS separation is achieved by laser excitation of the bound fluorophores, with excitation above a threshold level signaling the corresponding cell to be separated. MACS requires the antibody-labelled cells to be placed in a magnetic field and retained; unlabelled cells which are not bound are eluted, and labelled cells can be eluted once they are removed from the magnet, yielding separated cell populations (Tomlinson M J et al. J Tissue Eng 4: 2041731412472690). MACS is restricted to individual markers (although some kits use enzymatic removal of the microbeads, allowing the cells to be re-labelled with a subsequent antibody) and can be seen as a bulk method, i.e., there is no individual cell analysis. FACS, however, analyzes each individual cell, which can be tagged with multiple antibodies. This individual cell analysis means that while FACS can be more specific, it is significantly slower than MACS. Sorting that takes several hours by FACS can be achieved in less than 1 h by MACS (Tomlinson M J et al. J Tissue Eng 4: 2041731412472690).

Antibody-based methods of separation are the current gold standard for the selection of individual cell populations, and both FACS and MACS can be used to isolate cell populations to high purity. Despite this, there are still disadvantages to using these techniques. The conventional method for binding an antibody to a cell is a manual, open process. That is, antibodies and cells are added to a container and incubated on a rocking device. Following incubation, unbound antibody must be removed. This is traditionally accomplished by centrifugation, which pellets the cells, often resulting in physical damage and cell death. In addition, the isolation of a viable homogeneous population of cells that contain a unique intracellular marker can also be problematic, as the permeabilization steps required to stain the marker can damage cell membranes leading to cell death (Tomlinson M J et al. J Tissue Eng 4: 2041731412472690). Because these techniques involve an open process (i.e., exposed to the environment), microbial contamination of cell separation products remains an issue (Tomlinson M J et al. J Tissue Eng 4: 2041731412472690).

### Clinical Cell Therapy

The majority of separations currently performed for clinical cell therapy use cells isolated from tissues such as bone marrow and blood (Tomlinson M J et al. J Tissue Eng 4: 2041731412472690). These separations isolate mononuclear cells, including stem cells, and can be used to restore the hematopoietic system of a patient suffering from, for example, chronic myeloid leukaemia, following immune ablation therapy (Mancardi G and Saccardi R Lancet Neurol 2008; 7: 626-636). These separations primarily utilize systems based on centrifugation, such as apheresis (withdrawal of blood from a donor's body, removal of one or more blood components (e.g., plasma, platelets, white blood cells), and transfusion of the remaining blood back to the donor), as these technologies allow for the quick isolation of the large numbers of mononuclear cells needed for cell transplantation (Tomlinson M J et al. J Tissue Eng 4: 2041731412472690).

Standard FACS-based systems are not in clinical use for cell therapy, although some flow cytometers can be used for clinical diagnostics (Brown M and Wittwer C Clin Chem 2000; 46: 1221-1229). This is due, in part, to the difficulty in developing single-use sterile fluidics, the possibility of cross-contamination should multiuse fluidics be employed, and problems with batch-to-batch consistency (Tomlinson M J et al. J Tissue Eng 4: 2041731412472690).

Clinical cell separation is an established field with strict requirements and challenges and difficulties to overcome. The major requirement is to ensure that a consistent, sterile cell population is isolated. Microbial contamination of cell separation products could lead to the infection of the recipient patient, who, in many instances, is immunocompromised and unable to fight the infection. It is therefore imperative that clinical cell separation products are produced in closed (i.e., sterile, self-contained/closed to the environment) strict GMP conditions with stringent batch testing. Consistency of the isolated cell population is also very important so as to ensure that the recipient receives the required cell type and cell number during transplant (Tomlinson M J et al. J Tissue Eng 4: 2041731412472690).

Currently, the major challenge for clinical cell separation is the robust isolation of rare cell populations with multiple surface markers from a large initial pool of cells. For example, technologies based on centrifugation allow for the isolation of cells from a large initial cell number, and technologies based on MACS can isolate specific populations of cells. However, centrifugation, which pellets cells, often results in physical damage and cell death. In addition, because both centrifugation and MACS techniques involve an open process (i.e., exposed to the environment), microbial contamination of cell separation products remains an issue (Tomlinson M J et al. J Tissue Eng 4: 2041731412472690).

Therefore, a need exists for a system and method that is capable of performing a cell-selection process from initial source material to selected cell population while eliminating damage to, and contamination of, the selected cell population. The described invention provides an automated, closed, all-in-one system capable of effectively performing initial cell enrichment, cell labelling, and cell washing, resulting in direct delivery of cells selected based on size. The described invention reduces the risk of human error (i.e., automated), reduces the risk of contamination (i.e., closed system), and prevents damage of the selected cell population (i.e., does not require sedimentation/pelleting of cells).

### BRIEF SUMMARY OF THE INVENTION

According to one aspect, the described invention provides an automated, closed system for selecting a target cell population comprising: an input bag comprising a population of cells suspended in a physiological medium; a chamber embedded in a centrifuge rotor, into which the population of cells is passed; a capture particle injector comprising an agent adapted to identify a subpopulation of the population of cells; to select the subpopulation of the population of cells; and to be released from the subpopulation of the population of cells after the selection; an output bag comprising the released capture particle; the selected cells, or both; and a buffer bag comprising a wash buffer.

According to one embodiment, the capture particle injector comprises a capture particle adapted to recognize and bind to a cell surface marker on a surface of the subpopulation of the population of cells. According to another embodiment, the capture particle comprises a labeling agent that recognizes and binds to the cell surface marker.

According to one embodiment, the automated, closed system further comprises a labelling bag comprising a cell not bound to the capture particle and the capture particle not bound to a cell.

According to one embodiment, the agent is further conjugated to a bead.

According to one embodiment, the population of cells is a homogeneous cell population. According to another embodiment, the population of cells is a heterogeneous cell population.

According to one embodiment, the automated, closed system further comprises a pump.

According to one embodiment, the chamber is triangular-shaped.

According to one embodiment, the labeling agent adapted to recognize and bind to the cell-surface marker is an antibody.

According to one embodiment, the wash buffer is selected from the group consisting of Tris-buffered saline (TBS), phosphate buffered saline (PBS), Tris-buffered saline-tween-20 (TBST), phosphate-buffered saline-tween-20 (PBST), triethanolamine in PBS and a physiological medium. According to another embodiment, the physiological medium is selected from the group consisting of basal medium eagle (BME), Dulbecco's phosphate buffered saline (DPBS), Dulbecco's modified eagle medium (DMEM), DMEM-F12 media, F-10 nutrient mixture, Glasgow modified minimum essential medium (GMEM), Iscove's modified Delbucco's medium (IMDM), Leibovitz's L-15 medium, McCoy's 5A medium, MCDB 153 medium, media 199, minimal essential medium (MEM), minimal essential media alpha (MEMA), RPMI 1640 medium, CliniMACS® buffer, Hanks balanced salt saoltion (HBSS), TexMACs™ medium, and Waymouth's MB 752/1 medium.

According to one embodiment, the automated, closed system further comprises a lysing agent bag comprising a lysing agent that is effective to lyse the bead. According to another embodiment, the lysing agent bag comprises a calcium chelating agent. According to another embodiment, the calcium chelating agent is selected from the group consisting of ethylenediaminetetraacetic acid (EDTA); ethylene glycol tetraacetic acid (EGTA); 1,2-bis(o-aminophenoxy)ethane-N,N,N′,N′-tetraacetic acid (BAPTA); deferoxamine mesylate, iron chelator IV, 21H7; and N,N,N′,N′-tetrakis(2-pyridylmethy)ethane-1,2-diamine (TPEN). According to another embodiment, the calcium chelating agent is ethylenediaminetetraacetic acid (EDTA).

According to one embodiment, the bead is comprised of a natural polymer. According to another embodiment, the natural polymer is selected from the group consisting of alginate, an alginate derivative, agarose, cross-linked agarose (Sepharose®), collagen and chitosan. According to another embodiment, the natural polymer is alginate. According to another embodiment, the bead comprises dextran coated with alginate. According to another embodiment, the bead is a microbead.

According to one embodiment, the antibody is selected from the group consisting of a monoclonal antibody, a polyclonal antibody and a synthetic antibody mimic. According to another embodiment, the monoclonal antibody is selected from the group consisting of a synthetic antibody and an engineered antibody. According to another embodiment, the synthetic antibody is a recombinant antibody. According to another embodiment, the recombinant antibody is selected from the group consisting of a single-chain variable fragment (scFv) antibody, a nucleic acid aptamer and non-immunoglobulin protein scaffold. According to another embodiment, the engineered antibody is selected from the group consisting of a chimeric antibody and a humanized antibody.

According to another aspect, the described invention provides a method for isolating a substantially pure population of cells from a heterogeneous cell suspension using the automated, closed system according to claim 1, comprising: mixing a heterogeneous cell population with capture particles in a chamber embedded in a centrifuge rotor while the rotor is in motion and a counterflow in the chamber produces an opposing force within the chamber, wherein the capture particles comprise a bead conjugated to an agent that recognizes a specific cell surface marker; binding cells to the capture particles in the chamber embedded in the centrifuge rotor while the rotor is in motion and the counterflow produces an opposing force within the chamber, wherein the cells bound to capture particles express the specific cell-surface marker recognized by the agent that recognizes the specific cell surface marker; passing a wash buffer through the chamber embedded in the centrifuge rotor while the rotor is in motion and the counterflow produces an opposing force within the chamber, wherein the wash buffer removes unbound cells and unbound capture particles from the chamber; collecting the cells bound to the agent that recognizes the specific cell surface marker, wherein the cells bound to the agent that recognizes the specific cell surface marker are enriched relative to the heterogeneous cell suspension; and dissociating the cells in d. from the agent that recognizes the specific cell surface marker, wherein the method is effective to: reduce the risk of contamination of the collected cells; reduce damage to the collected cells; maintain viability of the collected cells; or a combination thereof.

According to one embodiment, the bead is comprised of a natural polymer. According to another embodiment, the natural polymer is selected from the group consisting of alginate, an alginate derivative, agarose, cross-linked agarose (Sepharose®), collagen and chitosan. According to another embodiment, the natural polymer is alginate. According to another embodiment, the bead comprises dextran coated with alginate. According to another embodiment, the bead is a microbead.

According to one embodiment, the agent that recognizes the specific cell surface marker is an antibody. According to another embodiment, the antibody is selected from the group consisting of a monoclonal antibody, a polyclonal antibody, an engineered antibody, and a synthetic antibody mimic. According to another embodiment, the synthetic antibody mimic is a recombinant antibody. According to another embodiment, the recombinant antibody is selected from the group consisting of a single-chain variable fragment (scFv) antibody, a nucleic acid aptamer and a non-immunoglobulin protein scaffold. According to another embodiment, the engineered antibody is selected from the group consisting of a chimeric antibody and a humanized antibody.

According to one embodiment, the wash buffer is selected from the group consisting of Tris-buffered saline (TBS), phosphate buffered saline (PBS), Tris-buffered saline-tween-20 (TBST), phosphate-buffered saline-tween-20 (PBST), triethanolamine in PBS and a physiological medium. According to another embodiment, the physiological medium is selected from the group consisting of basal medium eagle (BME), Dulbecco's phosphate buffered saline (DPBS), Dulbecco's modified eagle medium (DMEM), DMEM-F12 media, F-10 nutrient mixture, Glasgow modified minimum essential medium (GMEM), Iscove's modified Delbucco's medium (IMDM), Leibovitz's L-15 medium, McCoy's 5A medium, MCDB 153 medium, media 199, minimal essential medium (MEM), minimal essential media alpha (MEMA), RPMI 1640 medium, CliniMACS® buffer, Hanks balanced salt saoltion (HBSS), TexMACs™ medium, and Waymouth's MB 752/1 medium.

According to one embodiment, the method further comprises adding a lysing agent to the chamber embedded in the centrifuge rotor while the rotor is in motion, the counterflow produces an opposing force within the chamber, wherein the lysing agent lyses the bead.

According to one embodiment, the method further comprises passing a wash buffer through the chamber embedded in the centrifuge rotor while the rotor is in motion, the counterflow producing an opposing force within the chamber, wherein the wash buffer removes the lysing agent and the lysed bead.

According to one embodiment, the lysing agent is a calcium chelating agent. According to another embodiment, the calcium chelating agent is selected from the group consisting of ethylenediaminetetraacetic acid (EDTA); ethylene glycol tetraacetic acid (EGTA); 1,2-bis(o-aminophenoxy)ethane-N,N,N′,N′-tetraacetic acid (BAPTA); deferoxamine mesylate, iron chelator IV, 21H7; and N,N,N′,N′-tetrakis(2-pyridylmethy)ethane-1,2-diamine (TPEN). According to another embodiment, the calcium chelating agent is ethylenediaminetetraacetic acid (EDTA).

According to one embodiment, the collecting is performed by stopping the motion of the centrifuge rotor, increasing rate of the counterflow or a combination thereof.

According to one embodiment, the contamination is selected from the group consisting of bacterial contamination, viral contamination, fungal contamination and cellular debris.

According to one embodiment, the damage is selected from the group consisting of cellular swelling, fat accumulation, metabolic failure, structural damage/deterioration and apoptosis.

According to one embodiment, the dissociating is performed with a dissociation solution. According to another embodiment, the dissociation solution is selected from the group consisting of a pH solution, an ionic strength solution, a denaturing solution and an organic solution. According to another embodiment, the pH solution is selected from the group consisting of 100 mM glycine-HCl, pH 2.5-3.0; 100 mM citric acid, pH 3.0; 50-100 mM trimethylamine or triethanolamine, pH 11.5; and 150 mM ammonium hydroxide, pH 10.5. According to another embodiment, the ionic strength solution is selected from the group consisting of 3.5-4.0 M magnesium chloride, pH 7.0 in 10 mM Tris; 5 M lithium chloride in 10 mM phosphate buffer, pH 7.2; 2.5 M sodium iodide, pH 7.5; and 0.2-3.0 M sodium thiocyanate. According to another embodiment, the denaturing solution is selected from the group consisting of 2-6 M guanidine-HCl; 2-8 M urea; 1% deoxycholate; and 1% sodium dodecyl sulfate (SDS). According to another embodiment, the organic solution is selected from the group consisting of 10% dioxane and 50% ethylene glycol, pH 8-11.5.

According to one embodiment, the method further comprises isolating the labeled targeted subpopulation of cells from the heterogeneous cell population based on size, density, buoyancy or a combination thereof of the labeled targeted subpopulation of cells, wherein the capture particle is effective to alter size, density, buoyancy or a combination thereof of the target cell, and binding of the capture particle comprising the agent that recognizes and binds specifically to the target subpopulation of cells within the heterogeneous cell population is effective to change at least one of size, density and buoyancy of each target cell relative to an unlabeled cell in the heterogeneous cell population.

According to another aspect, the described invention provides a method for efficient viral-mediated gene transfer in mammalian cells comprising: Providing a first input bag containing a mammalian cell population and a second input bag containing a transduction buffer comprising a concentrated viral vector that is packaged with genetic material foreign to the mammalian cell population; Adding the first input bag containing the mammalian cell population and the transduction buffer comprising a concentrated viral vector that is packaged with genetic material foreign to the mammalian cell population to a chamber embedded in a centrifuge rotor while the rotor is in motion and a counterflow in the chamber produces an opposing force within the chamber; Incubating the mammalian cell population with the concentrated viral vector packaged with genetic material foreign to the mammalian cell population by circulating the transduction buffer comprising the concentrated viral vector that is packaged with the genetic material of interest around the cells, wherein the incubating is effective to transfer genetic material from the viral vector to a subpopulation of the mammalian cell population to form a transfected subpopulation of mammalian cells; selectively labeling the transfected subpopulation of mammalian cells by incubating the mammalian cell population with a capture particle comprising an agent that recognizes and binds specifically a cell antigen expressed selectively by the transfected subpopulation within the heterogeneous cell population; binding the capture particle comprising the agent to the targeted population of cells, to form a labeled transfected subpopulation of cells; passing a wash buffer through the chamber embedded in the centrifuge rotor while the rotor is in motion and the counterflow produces an opposing force within the chamber, wherein the wash buffer removes unbound cells and unbound capture particles from the chamber; collecting in an output bag the transfected subpopulation of cells bound to the capture particle comprising the agent that recognizes the specific cell surface marker so that the cells bound to the agent that recognizes the specific cell surface marker are enriched relative to the heterogeneous cell suspension; and dissociating the cells in (f) from the agent that recognizes the specific cell surface marker, wherein the method is effective to: reduce the risk of contamination of the collected cells; reduce damage to the collected cells; maintain viability of the collected cells; or a combination thereof.

According to one embodiment, binding of the capture particle comprising the agent that recognizes and binds specifically to the transfected subpopulation of cells within the heterogeneous cell population is effective to change at least one of size, density and buoyancy of each transfected cell compared to an unlabeled cell in the heterogeneous cell population.

According to one embodiment, the method further comprises adding a lysing agent to the chamber embedded in the centrifuge rotor while the rotor is in motion and the counterflow produces an opposing force within the chamber, wherein the lysing agent lyses the bead; and passing a wash buffer through the chamber embedded in the centrifuge rotor while the rotor is in motion and the counterflow produces an opposing force within the chamber, wherein the wash buffer removes the lysing agent and the lysed bead.

These and other advantages of the invention will be apparent to those of ordinary skill in the art by reference to the following detailed description and the accompanying drawings.

### BRIEF DESCRIPTION OF THE DRAWINGS

FIG. 1 shows a schematic representation of the described invention.

FIG. 2 shows a schematic representation of the principles of counterflow centrifugation (from Beckman Coulter's Optimizing Cell Separation with Beckman Coulter's Centrifugal Elutriation System).

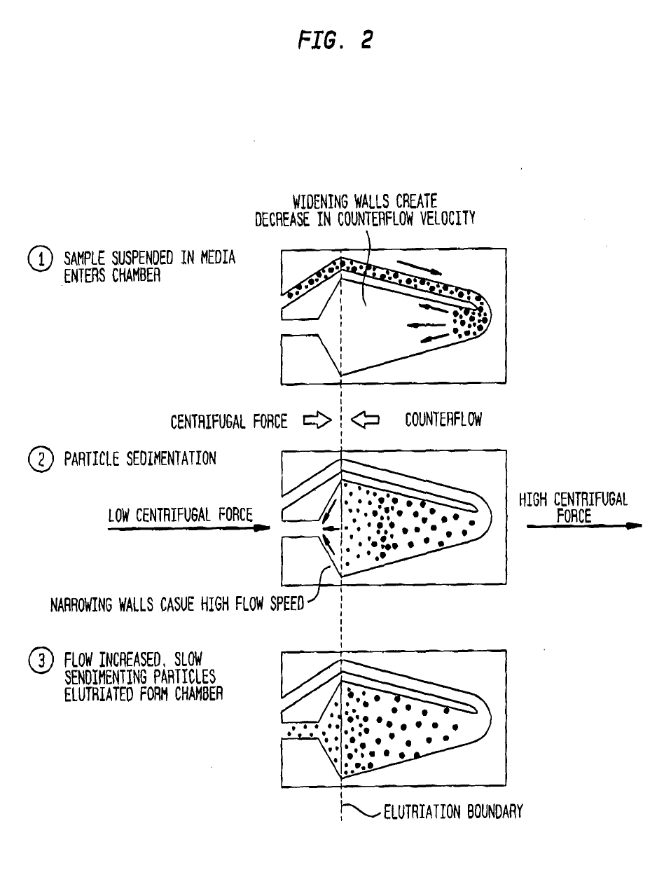

FIG. 3 shows a schematic representation of the method of the described invention.

FIG. 4 shows a schematic representation of a process of transduction performed using the described invention.

### DETAILED DESCRIPTION OF THE INVENTION

### Glossary

The term “ablation”, as used herein, refers to removal of a body part or destruction of its function, for example, by surgical procedure or morbid process, or the presence or application of a noxious substance. The terms “immune ablation”, “immunoablation”, “immune ablation therapy”, “immunoablation therapy”, “immune ablation treatment” and “immunoablation treatment” as used herein, refer to the systematic destruction of a patient's immune competence, often used, for example, to prepare a patient for organ transplantation or to treat a refractory autoimmune disease, especially when followed by immunoreconstruction by transplantation of cells including, but not limited to, autologous stem cells.

The term “affinity” as used herein, refers to a thermodynamic expression of the strength of interaction between a single antigen binding site and a single antigenic determinant (e.g., antibody and antigen). Affinity is expressed as the association constant, K. The term “high affinity” as used herein, refers to a strong intermolecular force of attraction (i.e., high/strong binding). The term “low affinity” as used herein, refers to a weak intermolecular force of attraction (i.e., low/weak binding).

The term “antibody”, as used herein, includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof.

Antibodies are serum proteins the molecules of which possess small areas of their surface that are complementary to small chemical groupings on their targets. These complementary regions (referred to as the antibody combining sites or antigen binding sites) of which there are at least two per antibody molecule, and in some types of antibody molecules ten, eight, or in some species as many as 12, may react with their corresponding complementary region on the antigen (the antigenic determinant or epitope) to link several molecules of multivalent antigen together to form a lattice.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In normal serum, 60% of the molecules have been found to have κ determinants and 30 percent λ. Many other species have been found to show two kinds of light chains, but their proportions vary. For example, in the mouse and rat, λ chains comprise but a few percent of the total; in the dog and cat, κ chains are very low; the horse does not appear to have any κ chain; rabbits may have 5 to 40% λ, depending on strain and b-locus allotype; and chicken light chains are more homologous to λ than κ.

In mammals, there are five classes of antibodies, IgA, IgD, IgE, IgG, and IgM, each with its own class of heavy chain-α (for IgA), δ (for IgD), ε (for IgE), γ (for IgG) and μ (for IgM). In addition, there are four subclasses of IgG immunoglobulins (IgG1, IgG2, IgG3, IgG4) having γ1, γ2, γ3, and γ4 heavy chains respectively. In its secreted form, IgM is a pentamer composed of five four-chain units, giving it a total of 10 antigen binding sites. Each pentamer contains one copy of a J chain, which is covalently inserted between two adjacent tail regions.

All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human λ light chains with the heavy chain variable region (VH) domain of a mouse monoclonal antibody reactive with an antigen of interest; (ii) selecting half-human Fabs on that antigen (iii) using the selected K light chain genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1, λ antibody molecule in the mouse myeloma.

The term “antigen” and its various grammatical forms refers to any substance that can stimulate the production of antibodies and/or can combine specifically with them. The term “antigenic determinant” or “epitope” as used herein refers to an antigenic site on a molecule. Sequential antigenic determinants/epitopes essentially are linear chains. In ordered structures, such as helical polymers or proteins, the antigenic determinants/epitopes essentially would be limited regions or patches in or on the surface of the structure involving amino acid side chains from different portions of the molecule which could come close to one another. These are conformational determinants.

The term “antigen presenting cells (APCs)”, as used herein, refers to cells of the immune system used for presenting antigen to T cells. APCs include, but are not limited to, dendritic cells, monocytes, macrophages, marginal zone Kupffer cells, microglia, Langerhans cells, T cells, and B cells. Antigen-presenting cells display several types of protein molecules on their surface, including, but not limited to, major histocompatibility complex (MHC) proteins; costimulatory proteins; and cell-cell adhesion molecules.

The term “apheresis”, as used herein refers to withdrawal of blood from a donor's body, removal of one or more blood components (e.g., plasma, platelets, white blood cells, etc.), and transfusion of the remaining blood back into the donor.

The term “associate”, and its various grammatical forms as used herein refers to joining, connecting, or combining to, either directly, indirectly, actively, inactively, inertly, non-inertly, completely or incompletely. Associated includes “connected.”

The term “automate”, as used herein, refers to running or operating a device, a system, etc., by using machines, computers, etc., instead of using manual operation.

The term “bind” means to combine with.

The term “bind specifically”, as used herein, refers to the principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, binding specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens. The term “multi-specificity”, as used herein, refers to binding of an antibody to more than one antigen.

The term “bond”, or “chemical bonds”, or “bonded”, are used interchangeably herein and refer to an attraction between atoms, alone or part of a larger molecule, that enables the formation of larger compounds. The term bond is inclusive of all different strengths and types, including covalent bonds, ionic bonds, halogen bonding, hydrogen bonds, van der waals forces, and hydrophobic effects.

The term “cell-surface marker”, as used herein, refers to an antigenic determinant or epitope found on the surface of a specific type of cell. Cell surface markers can facilitate the characterization of a cell type, its identification, and its isolation. Cell sorting techniques are based on cellular biomarkers where a cell surface marker(s) may be used for either positive selection or negative selection, i.e., for inclusion or exclusion, from a cell population.

The term “chimeric antibodies” as used herein refers to antibodies in which the rodent antibody constant region is swapped out for sequences found in human antibody.

The term “closed system” or “isolated system”, as used herein, refers to a physical system that is isolated from its surroundings, allows no exchange of matter or energy with its surroundings, and is not subject to any force whose source is external to the system.

The term “cluster of differentiation (CD)”, as used herein, refers to a defined subset of cell surface molecules, that identify cell type and stage of differentiation, and which are recognized by antibodies. CD molecules can act in numerous ways, often acting as receptors or ligands; by which a signal cascade is initiated, altering the behavior of the cell. Some CD proteins do not play a role in cell signaling, but have other functions, such as cell adhesion. Generally, a proposed surface molecule is assigned a CD number once two specific monoclonal antibodies (mAb) are shown to bind to the molecule. If the molecule has not been well-characterized, or has only one mAb, the molecule usually is given the provisional indicator “w.” More than 350 CD molecules have been identified for humans.

CD molecules are utilized in cell sorting by various methods, including flow cytometry. Cell populations usually are defined using a “+” or a “−” symbol to indicate whether a certain cell fraction expresses (“+”) or lacks (“−”) a CD molecule. For example, a “CD34+, CD31−” cell is one that expresses CD34, but not CD31. Table 1 shows commonly used markers employed by skilled artisans to identify and characterize differentiated white blood cell types:

| Type of Cell | CD Markers |
|---|---|
| Stem cells | CD34+, CD31− |
| All leukocyte groups | CD45+ |
| Granulocyte | CD45+, CD15+ |
| Monocyte | CD45+, CD14+ |
| T lymphocyte | CD45+, CD3+ |
| T helper cell | CD45+, CD3+, CD4+ |
| Cytotoxic T cell | CD45+, CD3+, CD8+ |
| B lymphocyte | CD45+, CD19+ or CD45+, CD20+ |
| Thrombocyte | CD45+, CD61+ |
| Natural killer cell | CD16+, CD56+, CD3− |

CD molecules used in defining leukocytes are not exclusively markers on the cell surface. Most CD molecules have an important function, although only a small portion of known CD molecules have been characterized.

The term “complementarity determining region” as used herein refers to immunoglobulin (Ig) hypervariable domains that determine specific antibody (Ab) binding. There are 6 CDRs in both variable regions of light (VL) and heavy chains (VH) with background variability on each side of the CDRs. Antibodies (Abs) of different specificities can assemble identical VL domains with different VH domains. The framework sequences between CDRs can be similar or identical.

The term “conjugate” or “conjugated”, as used herein, refers to reversibly binding, coupling or connecting one substance with another substance (e.g., an antibody to a bead).

The term “connected”, as used herein, refers to is being joined, linked, or fastened together in close association. For example, in the context of a chemical compound the term “connected to” refers to the attraction or connection between two atoms or molecules via direct or indirect chemical bonds.

The term “medium”, “culture medium”, and “physiological medium”, as used herein, refers generally to any preparation used for the cultivation of living cells. A “cell culture” refers to cells cultivated in vitro.

The term “cytometry”, as used herein, refers to a process in which physical and/or chemical characteristics of single cells, or by extension, of other biological or nonbiological particles in roughly the same size or stage, are measured. In flow cytometry, the measurements are made as the cells or particles pass through the measuring apparatus (a flow cytometer) in a fluid stream. A cell sorter, or flow sorter, is a flow cytometer that uses electrical and/or mechanical means to divert and collect cells (or other small particles) with measured characteristics that fall within a user-selected range of values.

The term “derivative”, as used herein, refers to a compound that may be produced from another compound of similar structure in one or more steps. A “derivative” or “derivatives” of a peptide or a compound retains at least a degree of the desired function of the peptide or compound. Accordingly, an alternate term for “derivative” may be “functional derivative.” Derivatives can include chemical modifications of the peptide, such as akylation, acylation, carbamylation, iodination or any modification that derivatizes the peptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine. Also included as derivatives or analogues are those peptides that contain one or more naturally occurring amino acid derivative of the twenty standard amino acids, for example, 4-hydroxyproline, 5-hydroxylysine, 3-methylhistidine, homoserine, ornithine or carboxyglutamiate, and can include amino acids that are not linked by peptide bonds. Such peptide derivatives can be incorporated during synthesis of a peptide, or a peptide can be modified by wellknown chemical modification methods (see, e.g., Glazer et al., Chemical Modification of Proteins, Selected Methods and Analytical Procedures, Elsevier Biomedical Press, New York (1975)).

The term “differential label” as used herein, generally refers to a stain, dye, marker, antibody or antibody-dye combination, or intrinsically fluorescent cell-associated molecule, used to characterize or contrast components, small molecules, macromolecules, e.g., proteins, and other structures of a single cell or organism. The term “dye” (also referred to as “fluorochrome” or “fluorophore”) as used herein refers to a component of a molecule which causes the molecule to be fluorescent. The component is a functional group in the molecule that absorbs energy of a specific wavelength and re-emits energy at a different (but equally specific) wavelength. The amount and wavelength of the emitted energy depend on both the dye and the chemical environment of the dye. Many dyes are known, including, but not limited to, FITC, R-phycoerythrin (PE), PE-Texas Red Tandem, PE-Cy5 Tandem, propidium iodem, EGFP, EYGP, ECF, DsRed, allophycocyanin (APC), PerCp, SYTOX Green, courmarin, Alexa Fluors (350, 430, 488, 532, 546, 555, 568, 594, 633, 647, 660, 680, 700, 750), Cy2, Cy3, Cy3.5, Cy5, Cy5.5, Cy7, Hoechst 33342, DAPI, Hoechst 33258, SYTOX Blue, chromomycin A3, mithramycin, YOYO-1, SYTOX Orange, ethidium bromide, 7-AAD, acridine orange, TOTO-1, TO-PRO-1, thiazole orange, TOTO-3, TO-PRO-3, thiazole orange, propidium iodide (PI), LDS 751, Indo-1, Fluo-3, DCFH, DHR, SNARF, Y66F, Y66H, EBFP, GFPuv, ECFP, GFP, AmCyan1, Y77W, S65A, S65C, S65L, S65T, ZsGreen1, ZsYellow1, DsRed2, DsRed monomer, AsRed2, mRFP1, HcRedl, monochlorobimane, calcein, the DyLight Fluors, cyanine, hydroxycoumarin, aminocoumarin, methoxycoumarin, Cascade Blue, Lucifer Yellow, NBD, PE-Cy5 conjugates, PE-Cy7 conjugates, APC-Cy7 conjugates, Red 613, fluorescein, FluorX, BODIDY-FL, TRITC, X¬rhodamine, Lissamine Rhodamine B, Texas Red, TruRed, and derivatives thereof.

The term “enriched” or enrichment”, as used herein, refers to increasing the concentration of a given substance above the initial concentration of the substance. For example, the term “cell enrichment”, as used herein, refers to increasing the concentration of a cell population above the initial concentration of the cell population.

The term “epitope” or antigenic determinant” or “epitope” means an antigenic site on a molecule. From Robert C. Ladner, Biotechnol. & Genetic Engineering Revs. 24 (1-30 (2007): Epitopes can be divided into linear epitopes (also known as continuous epitopes) and non-linear epitopes (also known as conformational or discontinuous epitopes). Linear epitopes persist after the protein is denatured or is in small peptide fragments. Conformational epitopes persist only in properly folded proteins or large folded fragments. “Epitope” can be modified or qualified in several ways. For example, there are “functional epitopes” (Sanchez-Madrid, et al., 1983), “structural epitopes” (Abraham, et al., 1985), “contact epitopes” (Jin, et al., 1992), “binding epitopes” (Bock, et al., 1985), “protective epitopes” (Seyer, et al., 1986), “neutralizing epitopes” (Wimmer, et al., 1984), “extracellular epitopes” (Khan, 2001), and “cytoplasmic epitopes” (Froehner, et al., 1983).

The term “flow cytometry”, as used herein, refers to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Analysis and differentiation of the cells is based on size, granularity, and whether the cells are carrying fluorescent molecules in the form of either antibodies or dyes. As the cell passes through the laser beam, light is scattered in all directions, and the light scattered in the forward direction at low angles (0.5-10°) from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population [Marion G. Macey, Flow cytometry: principles and applications, Humana Press, 2007].

The term “heterogeneous”, as used herein, refers to a substance comprising elements with various and dissimilar properties; not uniform in structure or composition. For example, a heterogeneous cell population comprises cells of different types (e.g., red blood cells, white blood cells, etc.).

The term “homogeneous” as used herein refers to a substance that is uniform in structure or composition.

The term “human antibody” as used herein refers to antibodies having variable and constant regions derived from human germline immunoglobulin sequences, but excludes from the definition antibodies in which CDR sequences derived from the germline of another mammalian species, such as a mouse, have been grafted onto human framework sequences.

The term “humanized antibodies” as used herein refers to antibodies in which rodent variable domain framework regions are swapped for human antibody sequences.

The term “immunoglobulin (Ig)”, as used herein, refers to a class of structurally related proteins, each consisting of two pairs of polypeptide chains, one pair of light (L) (low molecular weight) chains (k or l), and one pair of heavy (H) chains (g, a, m, d, and e), usually all four linked together by disulfide bonds. On the basis of the structural and antigenic properties of the H chains, Ig's are classified (in order of relative amounts present in normal human serum) as IgG, IgA, IgM, IgD, and IgE. Each class of H chain can associate with either k or l L chains. Subclasses of Ig's are based on differences in the H chains, and are referred to as IgG₁, etc.

When split by papain, IgG yields three pieces: the Fc piece, consisting of the C-terminal portion of the H chains, with no antibody activity but capable of fixing complement, and crystallizable; and two identical Fab pieces, each carrying an antigen-binding site and each consisting of an L chain bound to the remainder of an H chain.

All L chains are divided into a region of variable sequence (V_{L}) and one of constant sequence (C_{L}), each comprising about half the length of the L chain. The constant regions of all human L chains of the same type (κ or λ) are identical except for a single amino acid substitution, under genetic controls. H chains are similarly divided, although the V_{H} region, while similar in length to the V_{L} region, is only one-third or one-fourth the length of the C_{H} region. Binding sites are a combination of V_{L} and V_{H} protein regions. The large number of possible combinations of L and H chains make up the “libraries” of antibodies of each individual.

The term Ig includes, without limitation, naturally occurring and non-naturally occurring IgGs, polyclonal IgGs, monoclonal IgGs, chimeric IgGs, wholly synthetic IgGs, and fragments thereof.

The terms “isolate” and “separate” are used interchangeably herein to refer to placing, setting apart, or obtaining a cell, protein, molecule, substance, nucleic acid, peptide, or particle, in a form essentially free from contaminants or other materials with which it is commonly associated.

The term “labelling” as used herein, refers to a process of distinguishing a compound, structure, protein, peptide, antibody, cell or cell component by introducing an antibody, a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

The term “lectin” as used herein refers to a class of proteins that bind specifically to certain sugars.

The term “leukocyte”, as used herein, refers to a colorless cell (i.e., a white blood cell) that circulates in the blood and body fluids and is involved in counteracting foreign substances and disease. Leukocytes include, but are not limited to, lymphocytes, granulocytes, monocytes and macrophages.

The term “lymphocyte”, as used herein, refers to a small white blood cell formed in lymphatic tissue throughout the body and in normal adults making up about 22-28% of the total number of leukocytes in the circulating blood.

The term “mimetic” or “mimic”, as used herein, refers to chemicals containing chemical moieties that mimic the function of an antibody. For example, if an antibody binding site contains two charged chemical moieties having functional activity, a mimetic places two charged chemical moieties in a spatial orientation and constrained structure so that the charged chemical function is maintained in three-dimensional space.

The term “mononuclear cell” or “MNC”, as herein, refers to any cell that has a single round nucleus. Non-limiting examples include blood cells, such as lymphocytes, monocytes and dendritic cells.

The term “negative selection”, as used herein, refers to depletion or removal all cell types except for a cell type of interest, which remains.

The phrase “operatively linked” or “operably linked”, as used herein, refers to a linkage in which two or more protein domains are ligated or combined via recombinant DNA technology or chemical reaction such that each protein domain of the resulting fusion protein retains its original function.

The term “phenotype”, as used herein, refers to observable characteristics or physical traits (e.g., morphology, development, biochemical, physiological properties) of a cell or organism.

The term “positive selection”, as used herein, refers to the isolation of a target cell population.

The term “pure”, as used herein, refers to a cell, protein, molecule, substance, nucleic acid, peptide, or particle not mixed, adulterated or contaminated with any other substance or material.

The term “single chain variable fragment”, “single chain Fv” or “scFv” as used herein refers to antibody fragments comprising the V_{H} and V_{L} domains of an antibody. These domains are present in a single polypeptide chain. Generally, the Fv polypeptide further comprises a polypeptide linker between the V_{H} and V_{L} domains which enables the scFv to form the desired structure for antigen binding.

The term “substantially pure”, as used herein, refers to a purity of at least 75%, at least 80%, at least 85%, at least 90%, at least 95% or at least 99% as determined by an analytical protocol. Such protocols may include, for example, but are not limited to, FACS, HPLC, gel electrophoresis, chromatography, and the like.

The term “T lymphocyte” or “T-cell”, as used herein, generally refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. Two broad classes of lymphocytes are recognized: the B-lymphocytes (B-cells), which are precursors of antibody-secreting cells, and T-lymphocytes (T-cells).

T-lymphocytes derive from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. (Paul, W. E., “Chapter 1: The immune system: an introduction,” Fundamental Immunology, 4^{th} Edition, Ed. Paul, W. E., Lippicott-Raven Publishers, Philadelphia (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. Antibody and B-cell receptors evolved to bind to and to protect against microorganisms in extracellular fluids. In contrast, T cells recognize antigens on the surface of other cells and mediate their functions by interacting with, and altering, the behavior of these antigen-presenting cells (APCs). There are three main types of antigen-presenting cells in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an antigen-presenting cell (APC) that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the antigen-presenting cell (APC) for long enough to become activated. (“Chapter 24: The adaptive immune system,” Molecular Biology of the Cell, Alberts, B. et al., Garland Science, N Y, 2002).

T-cells are subdivided into two distinct classes based on the cell surface receptors they express. The majority of T cells express T cell receptors (TCR) consisting of α and β chains. A small group of T cells express receptors made of γ and δ chains. Among the α/β T cells are two important sublineages: those that express the coreceptor molecule CD4 (CD4+ T cells); and those that express CD8 (CD8+ T cells). These cells differ in how they recognize antigen and in their effector and regulatory functions.

CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells particularly CD8+ T cells, can develop into cytotoxic T-lymphocytes (CTLs) capable of efficiently lysing target cells that express antigens recognized by the CTLs. (Paul, W. E., “Chapter 1: The immune system: an introduction,” Fundamental Immunology, 4^{th} Edition, Ed. Paul, W. E., Lippicott-Raven Publishers, Philadelphia (1999)).

T cell receptors (TCRs) recognize a complex consisting of a peptide derived by proteolysis of the antigen bound to a specialized groove of a class II or class I MHC protein. The CD4+ T cells recognize only peptide/class II complexes while the CD8+ T cells recognize peptide/class I complexes. (Paul, W. E., “Chapter 1: The immune system: an introduction,” Fundamental Immunology, 4^{th} Edition, Ed. Paul, W. E., Lippicott-Raven Publishers, Philadelphia (1999)).

The TCR's ligand (i.e., the peptide/MHC protein complex) is created within antigen-presenting cells (APCs). In general, class II MHC molecules bind peptides derived from proteins that have been taken up by the APC through an endocytic process. These peptide-loaded class II molecules are then expressed on the surface of the cell, where they are available to be bound by CD4+ T cells with TCRs capable of recognizing the expressed cell surface complex. Thus, CD4+ T cells are specialized to react with antigens derived from extracellular sources. (Paul, W. E., “Chapter 1: The immune system: an introduction,” Fundamental Immunology, 4^{th} Edition, Ed. Paul, W. E., Lippicott-Raven Publishers, Philadelphia (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally nine amino acids in length, are bound into the class I MHC molecules and are brought to the cell surface, where they can be recognized by CD8+ T cells expressing appropriate receptors. This gives the T cell system, particularly CD8+ T cells, the ability to detect cells expressing proteins that are different from, or produced in much larger amounts than, those of cells of the remainder of the organism (e.g., vial antigens) or mutant antigens (such as active oncogene products), even if these proteins in their intact form are neither expressed on the cell surface nor secreted. (Paul, W. E., “Chapter 1: The immune system: an introduction,” Fundamental Immunology, 4^{th} Edition, Ed. Paul, W. E., Lippicott-Raven Publishers, Philadelphia (1999)).

T cells can also be classified based on their function as helper T cells; T cells involved in inducing cellular immunity; suppressor T cells; and cytotoxic T cells.

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4^{th} Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching (of the Ig class being expressed) either depend or are enhanced by the actions of T cell-derived cytokines. (Paul, W. E., “Chapter 1: The immune system: an introduction,” Fundamental Immunology, 4^{th} Edition, Ed. Paul, W. E., Lippicott-Raven Publishers, Philadelphia (1999)).

CD4+ T cells tend to differentiate into cells that principally secrete the cytokines IL-4, IL-5, IL-6, and IL-10 (T_{H2} cells) or into cells that mainly produce IL-2, IFN-γ, and lymphotoxin (T_{H1} cells). The T_{H2} cells are very effective in helping B-cells develop into antibody-producing cells, whereas the T_{H1} cells are effective inducers of cellular immune responses, involving enhancement of microbicidal activity of monocytes and macrophages, and consequent increased efficiency in lysing microorganisms in intracellular vesicular compartments. Although the CD4+ T cells with the phenotype of T_{H2} cells (i.e., IL-4, IL-5, IL-6 and IL-10) are efficient helper cells, T_{H1} cells also have the capacity to be helpers. (Paul, W. E., “Chapter 1: The immune system: an introduction,” Fundamental Immunology, 4^{th} Edition, Ed. Paul, W. E., Lippicott-Raven Publishers, Philadelphia (1999)). T-helper 1 (Th1) cells express at least one type of cell surface marker, including, but not limited to, chemokine (C-C motif) receptor 1 (CCR1), chemokine (C-C motif) receptor 5 (CCR5), cluster of differentiation 3 (CD3), cluster of differentiation 4 (CD4), chemokine (C-X-C motif) receptor 3 (CXCR3), interferon gamma receptor 1/cluster of differentiation 119 (IFN-γR1/CD119), interferon gamma receptor 2 (IFN-γR2), interleukin-12 receptor subunit beta-2 (IL-12Rβ2), interleukin-18 receptor alpha (IL-18Rα), and/or interleukin 27 receptor alpha/t cell cytokine receptor (IL-27Rα/TCCR). T-helper 2 (Th2) cells express at least one type of cell surface marker, including, but not limited to, chemokine (C-C motif) receptor 3 (CCR3), chemokine (C-C motif) receptor 4 (CCR4), chemokine (C-C motif) receptor 8 (CCR8), cluster of differentiation 3 (CD3), cluster of differentiation 4 (CD4), chemokine (C-X-C motif) receptor 4 (CXCR4), interferon gamma receptor 1/cluster of differentiation 119 (IFN-γR1/CD119), interferon gamma receptor 2 (IFN-γR2), interleukin-4 receptor alpha (IL-4Rα), interleukin-17 receptor beta (IL-17Rβ), interleukin-1 receptor 4 (IL-1R4), and/or thymic stromal lymphopoietin receptor (TSLPR).

A controlled balance between initiation and downregulation of the immune response is important to maintain immune homeostasis. Both apoptosis and T cell anergy (a tolerance mechanism in which the T cells are intrinsically functionally inactivated following an antigen encounter (Scwartz, R. H., “T cell anergy,” Annu. Rev. Immunol., 21: 305-334 (2003)) are important mechanisms that contribute to the downregulation of the immune response. A third mechanism is provided by active suppression of activated T cells by suppressor or regulatory CD4+T (Treg) cells. (Reviewed in Kronenberg, M. et al., “Regulation of immunity by self-reactive T cells,” Nature 435: 598-604 (2005)). CD4+ Tregs that constitutively express the IL-2 receptor alpha (IL-2Ra) chain (CD4+CD25+) are a naturally occurring T cell subset that are anergic and suppressive. (Taams, L. S. et l., “Human anergic/suppressive CD4+CD25+ T cells: a highly differentiated and apoptosis-prone population,” Eur. J. Immunol., 31: 1122-1131 (2001)). Depletion of CD4+CD25+ Tregs results in systemic autoimmune disease in mice. Furthermore, transfer of these Tregs prevents development of autoimmune disease. Human CD4+CD25+ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4+CD25+ T cells can be split into suppressive (CD25high) and nonsuppressive (CD25low) cells, according to the level of CD25 expression. A member of the forkhead family of transcription factors, FOXP3, has been shown to be expressed in murine and human CD4+CD25+ Tregs and appears to be a master gene controlling CD4+CD25+ Treg development. (Battaglia, M. et al., “Rapamycin promotes expansion of functional CD4+CD25+Foxp3+ regulator T cells of both healthy subjects and type 1 diabetic patients,” J. Immunol., 177: 8338-8347 (200)). Regulatory T-cells express at least one type of cell surface marker, including, but not limited to, cluster of differentiation 3 (CD3), cluster of differentiation 4 (CD4), cluster of differentiation 5 (CD5), cluster of differentiation 25 (CD25), cluster of differentiation 39 (CD39), cluster of differentiation 127 (CD127), cluster of differentiation 152 (CD152), cluster of differentiation 45RA (CD45RA), cluster of differentiation 45RO (CD45RO), cluster of differentiation 39 (CD39), cluster of differentiation 73 (CD73), cluster of differentiation 357 (CD357), cluster of differentiation 103 (CD103), cluster of differentiation 223 (CD223), cluster of differentiation 134 (CD134), cluster of differentiation 62L (CD62L), and/or cluster of differentiation 101 (CD101). Th9 cells express at least one type of cell surface marker, including, but not limited to, cluster of differentiation 3 (CD3), cluster of differentiation 4 (CD4), interleukin-4 receptor alpha (IL-4Ra), interleukin-17 receptor beta (IL-17Rβ), and/or transforming growth factor beta receptor II (TGF-βRII). Th17 cells express at least one type of cell surface marker, including, but not limited to, chemokine (C-C motif) receptor 4 (CCR4), chemokine (C-C motif) receptor 6 (CCR6), cluster of differentiation 3 (CD3), cluster of differentiation 4 (CD4), interleukin-1 receptor 1 (IL-1R1), interleukin-6 receptor alpha (IL-6Ro), interleukin-2 receptor (IL-21R), interleukin-23 receptor (IL-23R), and/or transforming growth factor beta receptor II (TGF-βRII).

The term “B lymphocyte” or “B-cell”, as used herein, refers to a short lived immunologically important lymphocyte that is not thymus dependent and is involved in humoral immunity. B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses. (Paul, W. E., “Chapter 1: The immune system: an introduction,” Fundamental Immunology, 4^{th} Edition, Ed. Paul, W. E., Lippicott-Raven Publishers, Philadelphia (1999)).

Cross-linkage dependent B-cell activation requires that the antigen express multiple copies of the epitope complementary to the binding site of the cell surface receptors because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes. (Paul, W. E., “Chapter 1: The immune system: an introduction,” Fundamental Immunology, 4^{th} Edition, Ed. Paul, W. E., Lippicott-Raven Publishers, Philadelphia (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4+ T-cells. The CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell. (Paul, W. E., “Chapter 1: The immune system: an introduction,” Fundamental Immunology, 4^{th} Edition, Ed. Paul, W. E., Lippicott-Raven Publishers, Philadelphia (1999)).

During cognate help for antibody production, the CD40 ligand is transiently expressed on activated CD4+T helper cells, and it binds to CD40 on the antigen-specific B cells, thereby tranducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in the pathogenic autoantibody production in human SLE patients. (Desai-Mehta, A. et al., “Hyperexpression of CD40 ligand by B and T cells in human lupus and its role in pathogenic autoantibody production,” J. Clin. Invest., 97(9): 2063-2073 (1996)).

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. For example, a mature B cell can be activated by an encounter with an antigen that expresses epitopes that are recognized by its cell surface immunoglobulin Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The soluble product of an activated B lymphocyte is immmunoglobulins (antibodies). The soluble product of an activated T lymphocyte is lymphokines.

The term “pro B-cell”, as used herein, refers to an early identifiable intermediate cell type in a series of developmental stages leading to the generation of mature B-cells. Human pro B-cells express at least one type of cell surface marker, including, but not limited to, cluster of differentiation 19 (CD19), cluster of differentiation 20 (CD20), cluster of differentiation 34 (CD34), cluster of differentiation 38 (CD38), and/or cluster of differentiation 45R (CD45R).

The term “pre-B-cell”, as used herein, refers to the immediate precursor cell of a mature B-cell. Human pre-B-cells express at least one type of cell surface marker, including, but not limited to, cluster of differentiation 19 (CD19), cluster of differentiation 20 (CD20), cluster of differentiation 38 (CD38), cluster of differentiation 40 (CD40), and/or cluster of differentiation 45R (CD45R).

The term “immature B-cell”, as used herein, refers to a cell produced in the bone marrow that migrates to secondary lymphoid tissues where it may develop into a mature B-cell. Human immature B-cells express at least one type of cell surface marker, including, but not limited to, cluster of differentiation 19 (CD19), cluster of differentiation 20 (CD20), cluster of differentiation 40 (CD40), cluster of differentiation 45R (CD45R), and/or immunoglobulin M (IgM).

The term “transitional B-cell”, as used herein, refers to an immature B-cell that has migrated to a secondary lymphoid tissue (e.g., spleen or lymph node). Human transitional 1 B-cells express at least one type of cell surface marker, including, but not limited to, cluster of differentiation 10 (CD10), cluster of differentiation 19 (CD19), cluster of differentiation 20 (CD20), cluster of differentiation 24 (CD24), cluster of differentiation 28 (CD28), and/or B-cell lymphoma 2 (BCL-2).

The term “naïve B-cell”, as used herein, refers to a B-cell that has not been exposed to an antigen. Human naïve B-cells express at least one type of cell surface marker, including, but not limited to, cluster of differentiation 19 (CD19), cluster of differentiation 20 (CD20), cluster of differentiation 23 (CD23), cluster of differentiation 38 (CD38), cluster of differentiation 40 (CD40), cluster of differentiation 150 (CD150), immunoglobulin M (IgM), and/or immunoglobulin D (IgD).

The term “memory B-cell”, as used herein, refers to a B-cell subtype that is formed within germinal centers following primary infection and are important in generating an antibody-mediated immune response in the case of re-infection. Human memory B-cells express at least one type of cell surface marker, including, but not limited to, cluster of differentiation 19 (CD19), cluster of differentiation 20 (CD20), cluster of differentiation 23 (CD23), cluster of differentiation 27 (CD27), cluster of differentiation 40 (CD40), immunoglobulin A (IgA), and/or immunoglobulin G (IgG).

The term “plasma cell”, as used herein, refers to a fully differentiated B-cell that produces a single type of antibody. Human plasma cells express at least one type of cell surface marker, including, but not limited to, cluster of differentiation 9 (CD9), cluster of differentiation 19 (CD19), cluster of differentiation 27 (CD27), cluster of differentiation 31 (CD31), cluster of differentiation 38 (CD38), cluster of differentiation 40 (CD40), cluster of differentiation 95 (CD95), and/or C-X-C chemokine receptor type 4 (CXCR-4).

The term “B-1 cell”, as used herein, refers to a sub-class of B-cell involved in the humoral immune response. They are not part of the adaptive immune system (i.e., they have no memory), but can generate antibodies against antigens and can act as antigen presenting cells. Human B-1 cells express at least one type of cell surface marker, including, but not limited to, cluster of differentiation 19 (CD19), cluster of differentiation 20 (CD20), cluster of differentiation 27 (CD27), immunoglobulin M (IgM), and/or immunoglobulin D (IgD).

The term “monocyte”, as used herein, refers to a large phagocytic white blood cell with a simple oval nucleus and clear, grayish cytoplasm. Monocytes are produced in the bone marrow and then enter the blood where they migrate to tissues (e.g., spleen, liver, lungs, and bone marrow tissue) where they mature into macrophages. Macrophages are the main scavenger cells of the immune system; engulfing apoptotic cells and pathogens to produce immune effector molecules which elicit an immune response. Monocytes/macrophages derived from humans express at least one type of cell surface marker, including, but not limited to, cluster of differentiation 14 (CD14), and/or cluster of differentiation 33 (CD33).

The term “dendritic cell”, as used herein, refers to antigen-presenting cells (APCs) that function to process antigen material and present it on the cell surface to T-cells. Dendritic cells are capable of presenting both major histocompatibility class I (MHC-I) and major histocompatibility class II (MHC-II) antigens. They act as messengers between the innate and the adaptive immune systems. Types of dendritic cells include, but are not limited to, myeloid (conventional) dendritic cells and plasmacytoid dendritic cells.

Myeloid dendritic cells derived from humans express at least one type of cell surface marker, including, but not limited to, cluster of differentiation 11c (CD11c), cluster of differentiation 123 (CD123), cluster of differentiation 1c/blood dendritic cell antigen-1 (CD1c/BDCA-1) and/or cluster of differentiation 141/blood dendritic cell antigen-3 (CD141/BDCA-3).

The term “plasmacytoid dendritic cell”, as used herein, refers to an innate immune cell that circulates in the blood and is found in peripheral organs. Plasmacytoid dendritic cells derived from humans express at least one type of cell surface marker, including, but not limited to, cluster of differentiation 304/blood dendritic cell antigen-4 (CD304/BDCA-4).

The term “open system”, as used herein, refers to a physical system in which material, energy, etc. can be gained from, or lost to, the surrounding environment.

The terms “saturate”, “saturation conditions” and “saturated conditions” are used interchangeably herein to refer to conditions in which one substance is united with another to the greatest possible extent. For example, filling of all available binding sites on an antibody molecule by its antigen. The terms “non-saturate”, “non-saturation conditions” and “non-saturated conditions” are used interchangeably herein to refer to conditions in which one substance is united with another to an extent less than the greatest possible extent, for example, not all available binding sites on an antibody are filled by its antigen.

The term “stem cell”, as used herein, refers to an undifferentiated cell having a high proliferative potential with the ability to self-renew that can generate daughter cells that can undergo terminal differentiation into more than one distinct cell phenotype. Types of stem cells include, but are not limited to, embryonic stem cells, non-embryonic somatic or adult stem cells and induced pluripotent stem cells (iPSCs).

Embryonic stem cells are derived from embryos that develop from eggs that have been fertilized in vitro. Embryonic stem cells derived from human subjects express at least one cell surface marker, including, but not limited to, stage-specific embryonic antigen-1 (SSEA-1), stage-specific embryonic antigen-3 (SSEA-3), stage-specific embryonic antigen-4 (SSEA-4), cluster of differentiation 324 (CD324/E-Cadherin), cluster of differentiation 90/thymus cell antigen-1 (CD90/Thy-1), cluster of differentiation 117/tyrosine-protein kinase kit/mast/stem cell growth factor receptor (CD117/c-KIT/SCFR), cluster of differentiation 326 (CD326), cluster of differentiation 9/multidrug resistance protein 1/transmembrane 4 superfamily/diphtheria toxin receptor-associated protein—27/24 kD protein (CD9/MRP1/TM4SF/DRAP-27/p24), cluster of differentiation 29 (CD29)/β1 integrin, cluster of differentiation 24/heat-stable antigen (CD24/HSA), cluster of differentiation 59 (CD59)/Protectin, cluster of differentiation 133 (CD133), cluster of differentiation 31/platelet endothelial cell adhesion molecule-1 (CD31/PECAM-1), cluster of differentiation 49f (CD49f)/Integrin α6/cluster of differentiation 29 (CD29), tumor rejection antigen 1-60 (TRA-1-60), tumor rejection antigen 1-81(TRA-1-81), Frizzled-5 (FZD5), Stem cell factor (SCF/c-Kit ligand), and/or Cripto/teratocarcinoma-derived growth factor-1 (TDGF-1).

Somatic or adult stem cells are undifferentiated cells found among differentiated cells in a tissue or organ. These cells can renew themselves and can differentiate to yield some or all of the major specialized cell types of the tissue or organ of origin. The primary role of somatic/adult stem cells is to maintain and repair the tissue in which they are found. Somatic/adult stem cells include, but are not limited to, hematopoietic stem cells and mesenchymal stem cells.

The term “hematopoietic stem cell (HSC)” as used herein, refers to a cell isolated from blood or from bone marrow that can renew itself, differentiate to a variety of specialized cells, mobilize out of the bone marrow into the circulating blood, and undergo programmed cell death (apoptosis). Hematopoietic stem cells derived from human subjects express at least one type of cell surface marker, including, but not limited to, cluster of differentiation 34 (CD34), cluster of differentiation 38 (CD38), cluster of differentiation 45RA (CD45RA), human leukocyte antigen-antigen D related (HLA-DR), cluster of differentiation 117/tyrosine-protein kinase kit/mast/stem cell growth factor receptor (CD117/c-KIT/SCFR), cluster of differentiation 59 (CD59), stem cell antigen-1 (Sca-1), cluster of differentiation 90/thymus cell antigen-1 (CD90/Thy-1), and/or C-X-C chemokine receptor type 4 (CXCR-4).

The term “mesenchymal stem cell (MSC)”, as used herein, refers to a multipotent stromal cell that can differentiate into a variety of cells, including, but not limited to, osteoblasts (bone cells), chondrocytes (cartilage cells), myocytes (muscle cells) and adipocytes (fat cells). In vivo, MSCs associate with HSCs, exerting a key regulatory effect on early stages of hematopoiesis. MSCs also enter differentiation pathways to replenish mature osteoblasts, adipocytes and hemo-supportive stroma in bone marrow. MSCs are innervated by sympathetic nervous system fibers and mediate neural control of hematopoiesis. Mesenchymal stem cells derived from human subjects do not express a single specific identifying marker but have been shown to express at least one type of cell surface marker, including, but not limited to, stromal precursor antigen-1 (Stro-1), stage-specific embryonic antigen-4 (SSEA-4), cluster of differentiation 70 (CD70), cluster of differentiation 271 (CD271), cluster of differentiation 200 (CD200), cluster of differentiation 146/melanoma cell adhesion molecule (CD146/MCAM), cluster of differentiation 73 (CD73)/5′-nucleotidase, cluster of differentiation 90/thymus cell antigen-1 (CD90/Thy-1), cluster of differentiation 105 (CD105)/endoglin, cluster of differentiation 106/vascular cell adhesion molecule-1 (CD106/VCAM-1), Ganglioside GD2, Frizzled-9 (FZD9), Tissue non-specific alkaline phosphatase (TNAP), and/or Sushi domain containing 2 (SUSD2).

The term “induced pluripotent stem cell (iPSC)”, as used herein, refers to a type of pluripotent stem cell that can be generated directly from adult cells. These cells are genetically reprogrammed to an embryonic stem cell-like state by being forced to express genes and factors important for maintaining the defining properties of embryonic stem cells. Induced pluripotent stem cells derived from human subjects express at least one type of cell surface marker, including, but not limited to, stage-specific embryonic antigen-1 (SSEA-1), stage-specific embryonic antigen-4 (SSEA-4), alkaline phosphatase, octamer-binding transcription factor 3/4 (Oct-3/4), homeobox protein Nanog (NANOG), sex determining region Y-box 2 (Sox2), Krueppel-like factor 4 (KLF4), tumor rejection antigen 1-60 (TRA-1-60), tumor rejection antigen 1-81(TRA-1-81), and/or tumor rejection antigen 2-54 (TRA-2-54).

The term “subject” or “individual” or “patient” are used interchangeably herein to refer to a member of an animal species of mammalian origin, including but not limited to, mouse, rat, cat, goat, sheep, horse, hamster, ferret, pig, dog, platypus, guinea pig, rabbit and a primate, such as, for example, a monkey, ape, or human.

The term “target cell” or “targeted cell”, as used herein, refers to a cell that has a specific cell-surface marker that reacts with or binds to a specific antibody.

According to one aspect, the described invention provides an automated, closed system for effectively labelling a target population of cells, washing the target population of cells and enriching for the labeled targeted population of cells, resulting in direct delivery of the targeted population of selected cells.

According to some embodiments, a population of cells suspended in a physiological medium can added alone to the system; according to some such embodiments, a labeling agent that recognizes a cell surface marker specifically and a capture particle adapted to bind to cells labeled with the labeling agent and to therefore selectively separate the subpopulation of labeled cells from the population of cells subsequently are added. According to some embodiments, the population of cells is labeled inside the automated, closed system of the described invention. According to some embodiments, the cells are labeled outside the automated, closed system of the described invention. According to some such embodiments, the population of cells plus the labeling agent that recognizes a cell surface marker specifically are combined outside the system; the population of cells plus the labeling agent that recognizes a cell surface marker specifically combined outside the system are then added to the system; and a capture particle adapted to bind to a subpopulation of cells labeled with the labeling agent that is effective to facilitate separation/selection of the subpopulation of labeled cells from the population of cells subsequently is then added to the system. According to some embodiments, the cells are labeled in the automated closed system of the described invention. According to some such embodiments, the population of cells plus the labeling agent that recognizes a cell surface marker specifically are combined inside the system; a capture particle adapted to bind to a subpopulation of cells labeled with the labeling agent that is effective to facilitate separation/selection of the subpopulation of labeled cells from the population of cells subsequently is added to the system. According to some embodiments, the population of cells, the labeling agent that recognizes a cell surface marker specifically, and the capture particle adapted to bind to cells labeled with the labeling agent and to therefore separate/select the subpopulation of labeled cells from the population of cells are added and combined within the system. In each case, the capture particle can be released from the captured cells after selection.

According to another aspect, the described invention can be used for efficient gene transfer in a population of mammalian cells. According to some embodiments, the gene transfer is mediated by a viral vector. According to some embodiments, the gene transfer is by transfection. According to some embodiments, the population of mammalian cells comprises labeled cells. According to some embodiments, the population of mammalian cells has been enriched for labeled cells by positively selecting for the desired cells using a labeling agent that recognizes a cell surface marker on a subpopulation of the population of mammalian cells. According to some embodiments, the population of mammalian cells comprises cells remaining after the labeled cells have been removed.

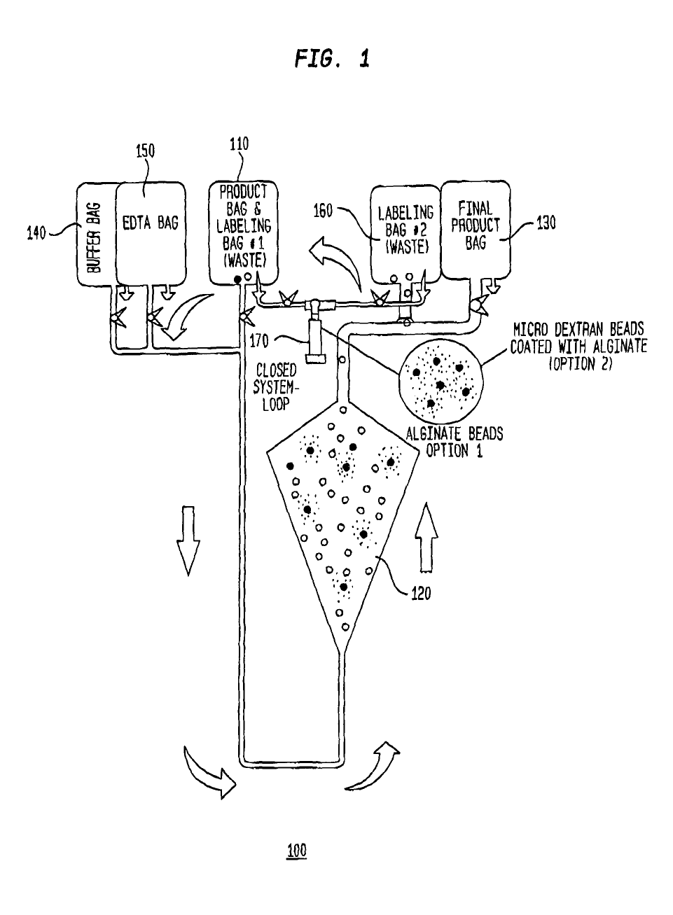

According to some embodiments, the automated, closed system comprises an initial product bag (110), a chamber embedded in a centrifuge rotor/chamber (120), a final product bag (130), a buffer bag (140), a labelling bag (160) and a capture particle injector (170). According to some embodiments, the automated, closed system (100) comprises an initial product bag (110), a chamber embedded in a centrifuge rotor/chamber (120), a final product bag (130), a buffer bag (140), a lysing agent bag (150), a labelling bag (160) and a capture particle injector (170) (See, e.g., FIG. 1). According to some embodiments, the automated, closed system comprises a pump.

According to one aspect, the described invention provides a method for labelling cells using the automated, closed system (100).

According to another aspect, the described invention provides a method for selecting/isolating cells using the automated, closed system (100).

According to another aspect, the described invention provides a method for labelling cells, washing, enriching/selecting the labeled cells, and directly delivering the selected cells using the automated, closed system (100).

According to some embodiments, the described invention provides a method for isolating a substantially pure population of cells from a heterogeneous cell suspension using the automated, closed system (100).

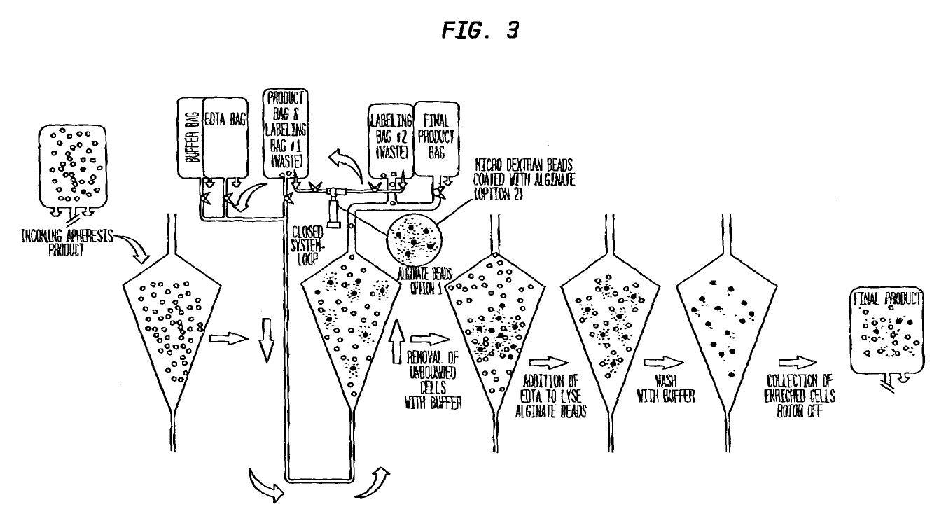

According to some embodiments, the method comprises passing a heterogeneous cell population and capture particles comprising an agent that recognizes and binds specifically to a cell into a chamber embedded in a centrifuge rotor/chamber (120) while the rotor is in motion (i.e., spinning). According to some embodiments, the agent that recognizes and binds specifically to a cell is an antibody that recognizes a specific cell surface marker and bind those cells within the heterogeneous cell population that contain the specific cell surface marker. According to some embodiments, the agent that recognizes and binds specifically to a cell is a lectin. According to some embodiments, the method comprises passing a wash buffer into the chamber embedded in the centrifuge rotor/chamber (120) while the rotor is in motion in order to remove unbound cells (i.e., cells that do not contain the specific cell surface marker). Next, according to some embodiments, the method comprises passing a lysing agent (e.g., EDTA) into the chamber embedded in the centrifuge rotor/chamber (120) while the rotor is in motion in order to lyse the capture particles and passing a wash buffer into the chamber embedded in the centrifuge rotor/chamber (120) while the rotor is in motion in order to remove the lysing agent and the lysed capture particles. According to some embodiments, the rotor is turned off (i.e., not spinning) and the cells bound to the agent that recognizes and binds specifically to a cell (i.e, enriched) are collected in a final product bag (130). According to some embodiments, flow rate of counterflow is increased and the cells bound to the agent that recognizes and binds specifically to a cell (i.e., enriched) are collected in a final product bag (130). According to some embodiments, the rotor is turned off (i.e., not spinning), flow rate of counterflow is increased and the cells bound to the agent that recognizes and binds specifically to a cell (i.e., enriched) are collected in a final product bag (130) (See, e.g., FIG. 3).

According to some embodiments, the described invention comprises an injector, which is adapted to add a capture particle to the chamber for selecting a subpopulation of cells. According to some embodiments, the injector is adapted to add the labeling agent and the capture particle to the chamber for selecting a subpopulation of cells. According to some embodiments, the injector is adapted to add the population of cells, the labeling agent, and the capture particle to the chamber for selecting a subpopulation of cells.

According to some embodiments, the chamber is of a shape useful to form a velocity gradient. According to some embodiments, the chamber is triangular-shaped.

According to some embodiments, the washing steps are performed by use of a wash buffer. Non-limiting examples of commercially-available wash buffers include Tris-buffered saline (TBS), phosphate buffered saline (PBS), Tris-buffered saline-tween-20 (TBST), phosphate-buffered saline-tween-20 (PBST), triethanolamine in PBS and physiological media. Physiological media includes, but is not limited to, basal medium eagle (BME), Dulbecco's phosphate buffered saline (DPBS), Dulbecco's modified eagle medium (DMEM), DMEM-F12 media, F-10 nutrient mixture, Glasgow modified minimum essential medium (GMEM), Iscove's modified Delbucco's medium (IMDM), Leibovitz's L-15 medium, McCoy's 5A medium, MCDB 153 medium, media 199, minimal essential medium (MEM), minimal essential media alpha (MEMA), RPMI 1640 medium, CliniMACS® buffer, Hanks balanced salt saoltion (HBSS), TexMACs™ medium, and Waymouth's MB 752/1 medium.

According to some embodiments, the described invention, which is automated, is effective to reduce the risk of human error.

According to some embodiments, the described invention is effective to reduce the risk of contamination, by, for example, a bacteria, a virus, a fungus, cellular debris and other unwanted materials with which the selected/isolated cells are commonly associated.

According to some embodiments, the described invention is effective to reduce damage to the selected/isolated cell population because the described invention does not require sedimentation/pelleting of cells. Types of damage include, but are not limited to, cellular swelling, fat accumulation, metabolic failure, structural damage/deterioration and apoptosis (i.e., cell death).

According to some embodiments, the described invention maintains viability (meaning the ability of a cell to live, grow, expand, etc.) of the selected/isolated cells.

According to some embodiments, the described invention maintains morphology of the selected/isolated cells.

According to some embodiments, the selected/isolated cell population is at least 75% pure. According to some embodiments, the selected/isolated cell population is at least 76% pure. According to some embodiments, the selected/isolated cell population is at least 77% pure. According to some embodiments, the selected/isolated cell population is at least 78% pure. According to some embodiments, the selected/isolated cell population is at least 79% pure. According to some embodiments, the selected/isolated cell population is at least 80% pure. According to some embodiments, the selected/isolated cell population is at least 81% pure. According to some embodiments, the selected/isolated cell population is at least 82% pure. According to some embodiments, the selected/isolated cell population is at least 83% pure. According to some embodiments, the selected/isolated cell population is at least 84% pure. According to some embodiments, the selected/isolated cell population is at least 85% pure. According to some embodiments, the selected/isolated cell population is at least 86% pure. According to some embodiments, the selected/isolated cell population is at least 87% pure. According to some embodiments, the selected/isolated cell population is at least 88% pure. According to some embodiments, the selected/isolated cell population is at least 89% pure. According to some embodiments, the selected/isolated cell population is at least 90% pure. According to some embodiments, the selected/isolated cell population is at least 91% pure. According to some embodiments, the selected/isolated cell population is at least 92% pure. According to some embodiments, the selected/isolated cell population is at least 93% pure. According to some embodiments, the selected/isolated cell population is at least 94% pure. According to some embodiments, the selected/isolated cell population is at least 95% pure. According to some embodiments, the selected/isolated cell population is at least 96% pure. According to some embodiments, the selected/isolated cell population is at least 97% pure. According to some embodiments, the selected/isolated cell population is at least 98% pure. According to some embodiments, the selected/isolated cell population is at least 99% pure.

According to some embodiments, the source of the selected/isolated cells includes, but is not limited to, skin, blood, bone marrow, brain, heart, liver, pancreas, lung, stomach, intestine, kidney, bladder, ovary, uterus, testis, thymus, adipose tissue and lymph node.

According to some embodiments, the selected/isolated cells are stem cells. Stem cells include, but are not limited to embryonic stem cells, somatic stem cells and induced pluripotent stem cells. Somatic stem cells include, but are not limited to, hematopoietic stem cells and mesenchymal stem cells.

According to some embodiments, the selected/isolated cells are mononuclear cells. Non-limiting examples of mononuclear cells include lymphocytes, monocytes and dendritic cells. Lymphocytes include, but are not limited to, T lymphocytes and B lymphocytes. T lymphocytes include, but are not limited to, T helper cells and regulatory T-cells. B lymphocytes include, but are not limited to, pro B-cells, pre B-cells, immature B-cells, transitional B-cells, naïve B-cells, memory B-cells, plasma cells and B-1 cells. Dendritic cells include, but are not limited to, myeloid (conventional) dendritic cells and plasmacytoid dendritic cells.

According to some embodiments, the population of cells is separated/isolated from components that normally accompany or interact with the population of cells as found in its natural environment (e.g., blood) based on size. Without being bound by theory, capture particles comprising the agent that recognizes and binds specifically to a cell can specifically bind to cell phenotypes to facilitate separation/isolation. Cells and capture particles are mixed and incubated within the automated, closed system of the described invention. Following incubation, cells bound to capture particles exhibit a larger size than unbound cells, allowing for separation within the system of the described invention. A wash is automatically performed within the closed system to remove unbound cells. Following the wash, a dissociation solution is automatically added within the closed system to remove the selected/isolated cells from the capture particles. The selected/isolated cells are automatically washed and volume is reduced within the closed system.

According to some embodiments, the dissociation solution is a pH solution. Non limiting examples of pH solutions include 100 mM glycine-HCl, pH 2.5-3.0; 100 mM citric acid, pH 3.0; 50-100 mM trimethylamine or triethanolamine, pH 11.5; and 150 mM ammonium hydroxide, pH 10.5. According to some embodiments, the dissociation solution is an ionic strength solution. Ionic strength solutions include, but are not limited to, 3.5-4.0 M magnesium chloride, pH 7.0 in 10 mM Tris; 5 M lithium chloride in 10 mM phosphate buffer, pH 7.2; 2.5 M sodium iodide, pH 7.5; and 0.2-3.0 M sodium thiocyanate. According to some embodiments, the dissociation solution is a denaturing solution. Non-limiting examples of denaturing solutions include 2-6 M guanidine-HCl; 2-8 M urea; 1% deoxycholate; and 1% sodium dodecyl sulfate (SDS). According to some embodiments, the dissociation solution is an organic solution. Organic solutions include, but are not limited to, 10% dioxane and 50% ethylene glycol, pH 8-11.5.

According to some embodiments, the described invention utilizes a capture particle, for example, a pellet, an agglomerate, a crystal, or a bead. According to some embodiments, the capture particle comprises an agent that recognizes and binds specifically to a cell. According to some embodiments, the capture particle is a bead. According to some embodiments, the agent that recognizes and binds specifically to a cell is an antibody. According to some embodiments, the agent that recognizes and binds specifically to a cell is a lectin.

According to some embodiments, the bead is of a size useful for conjugation with the agent. Non-limiting examples include microbeads and nanobeads. According to some embodiments, the bead is biocompatible. According to some embodiments, the bead is of a shape useful for conjugation to the agent. According to some embodiments, the shape of the bead is irregular. According to some embodiments, the shape of the bead is uniform. Shapes of the bead include, but are not limited to, a sphere, an oval, a cylinder, a cube, a pyramid, a teardrop, a blob, a globule and the like. According to some embodiments, the bead is of a material useful for conjugation to the agent that recognizes and binds specifically to a cell surface marker. Non-limiting examples of materials include a polymer or a mixture of different polymers, including, but not limited to, poly(lactic-co-glycolic acid (PLGA), polyethylene glycol (PEG), polyorthoester, polyanhydride, polygutamic acid, polyaspartic acid and poly(lactide-co-caprolactone). According to some embodiments, the polymer is a synthetic polymer. Synthetic polymers include, but are not limited to, low density polyethylene (LDPE), high density polyethylene (HDPE), polypropylene (PP), polyvinyl chloride (PVC), polystyrene (PS), nylon, thermoplastic polyurethane (TPU), poly(vinyl alcohol), poly(ethylene glycol) and Teflon™. According to some embodiments, the polymer is a natural polymer. Natural polymers include, but are not limited to, alginate, alginate derivatives, agarose, Sepharose®, collagen and chitosan. Alginate derivatives include, but are not limited to, sodium alginate, amphiphilic alginate and cell-interactive alginate.

Alginate is a naturally occurring anionic polymer typically obtained from brown seaweed, and has been extensively investigated and used for many biomedical applications, due to its biocompatibility, low toxicity, relatively low cost, and mild gelation by addition of divalent cations such as Ca²⁺. Commercially available alginate is typically extracted from brown algae (Phaeophyceae), including Laminaria hyperborea, Laminaria digitata, Laminaria japonica, Ascophyllum nodosum, and Macrocystis pyrifera by treatment with aqueous alkali solutions, typically with sodium hydroxide (NaOH). The extract is filtered, and either sodium or calcium chloride is added to the filtrate in order to precipitate alginate. This alginate salt can be transformed into alginic acid by treatment with dilute HCl. After further purification and conversion, water-soluble sodium alginate power is produced.

Alginate can also be synthesized by bacteria. Bacterial biosynthesis by either Azotobacter oe Pseudomonas provides alginate with more defined chemical structures and physical properties than can be obtained from seaweed-derived alginate. The pathway of alginate biosynthesis is generally divided into (i) synthesis of precursor substrate; (ii) polymerization and cytoplasmic membrane transfer; (iii) periplasmic transfer and modification; and (iv) export through the outer membrane. Bacterial modification can enable production of alginate with tailor-made features and a broad range of biomedical applications.

Alginate is a family of linear copolymers containing blocks of (1,4)-linked β-D-mannuronate (M) and α-L-guluronate (G) residues. The blocks are composed of consecutive G residues (GGGGGG), consecutive M residues (MMMMMM), and alternating M and G residues (GMGMGM). Alginates extracted from different sources differ in M and G contents as well as the length of each block. Only the G-blocks of alginate are believed to participate in intermolecular cross-linking with divalent cations (e.g., Ca²⁺) to form hydrogels. The composition (i.e., M/G ratio), sequence, G-block length, and molecular weight are thus critical factors affecting the physical properties of alginate and its resultant hydrogels. The G-block content of Laminaria hyperborean stems is 60%, while other commercially available alginates have a G-block content in the range of 14.0-31.0%. The molecular weight of commercially available sodium alginates range between 32,000 and 400,000 g/mol. The viscosity of alginate solutions increases as pH decreases, and reaches a maximum around pH=3-3.5, as carboxylate groups in the alginate backbone become protonated and form hydrogen bonds.

Various alginate derivatives are available and are used in a range of biomedical applications. For example, amphiphilic alginate derivatives have been synthesized by introducing hydrophobic moieties (e.g., alkyl chains, hydrophobic polymers) to the alginate backbone. These derivatives can form self-assembled structures such as particles and gels in aqueous media. Amphiphilic derivatives of sodium alginate have been prepared by conjugation of long alkyl chains (i.e., dodecyl, octadecyl) to the alginate backbone via ester bond formation. Microparticles can be prepared from these derivatives by dispersion in a sodium chloride solution, this technique can allow encapsulation of proteins and their subsequent release by the addition of either surfactants that disrupt intermolecular hydrophobic junctions or esterases that hydrolyze the ester bond between alkyl chains and the alginate backbone. Dodecylamine can also be conjugated to the alginate backbone via amide linkage formation using 2-chloro-1-methylpyridinium iodide as a coupling reagent. Hydrogels prepared from this alginate derivative exhibit long-term stability in aqueous media, compared to those prepared from alginate derivatives with dodecyl ester, which are labile to hydrolysis. Water soluble, amphiphilic alginate derivatives grafted with cholesteryl groups can also be synthesized using N,N′-dicyclohexylcarbodiimide as a coupling agent and 4-(N,N′-dimethylamino)pyridine as a catalyst at room temperature. These derivatives form self-aggregates with a mean diameter of 136 nm in an aqueous sodium chloride solution. Sodium alginate can also be hydrophobically modified with poly(butyl methacrylate), leading to prolonged release of model drugs as compared with unmodified alginate gels

Cell-interactive alginates (i.e., alginate derivatives containing cell-adhesive peptides) can be prepared by chemically introducing peptides as side-chains, using carbodiimide chemistry to couple via the carboxylic groups of the sugar residues. Since alginate inherently lacks mammalian cell-adhesivity, appropriate ligands are necessary to promote and regulate cellular interactions, especially for cell culture and tissue engineering applications. Peptides including the sequence arginine-glycine-aspartic acid (RGD) have been extensively used as model adhesion ligands, due to the wide-spread presence of integrin receptors (e.g., αᵥβ₃, α₅β₁) for this ligand on various cell types. RGD containing peptides can be chemically coupled to the alginate backbone using water-soluble carbodiimide chemistry. A minimum concentration of RGD peptides in alginate gels is needed for the adhesion and growth of cells, and this minimum is likely cell type specific. For example, minimal concentrations for substantial adhesion of MC3T3-E1 and C2C12 cells to alginate gels in vitro have been reported as 12.5 and 10.0 μg/mg alginate, respectively. The affinity of the RGD peptide also plays an important role, and cyclic RGD peptides have been demonstrated to be more potent and are needed at lower concentrations than linear RGD peptides. Various peptides containing the DGEA (Asp-Gly-Glu-Ala) and YIGSR (Tyr-Ile-Gly-Ser-Arg) sequences derived from other extracellular matrix proteins have also been exploited to modify alginate gels and enhance the adhesive interactions with various cell types. For example, alginate has been modified with YIGSR peptides using water-soluble carbodiimide chemistry to promote neural cell adhesion (See, e.g., Lee K Y and Mooney D J Prog Polym Sci 2012 January; 37(1): 106-126).

Agarose is a purified linear galactan hydrocolloid isolated from agar or agar-bearing marine algae. It is a linear polymer consisting of alternating D-galactose and 3,6-anhydro-L-galactose units. As a gelling agent, agarose is used, for example, to separate nucleic acids electrophoretically; to demonstrate cross reaction in immunoelectrophoresis (IEP) and double diffusion plates in which antibody-antigen precipitin lines are studied; to make gel plates or overlays for cells in tissue culture; and to form a gel matrix (either beaded and/or crosslinked) which can be used, for example, in chromatographic separations.

Sepharose® is a cross-linked, beaded form of agarose primarily used for the chromatographic separation of biomolecules. Various grades and chemistries of Sepharose® are available which permit the selective binding of cysteine side chain for the immobilization of peptides. It can be combined with activation chemistries, such as cyanogen bromide (CNBr) and reductive amination of aldehydes, in order to immobilize antibodies, enzymes, proteins and peptides by way of covalent attachment.

Collagen is the most widely found protein in mammals and is the major provider of strength to tissue. A typical collagen molecule consists of three intertwined protein chains that form a helix. These molecules polymerize together to form collagen fibers of varying length, thickness, and interweaving pattern (e.g., some collagen molecules will form ropelike structures, while others will form meshes or networks). There are at least 15 different types of collagen, differing in their structure, function, location, and other characteristics. The predominant form used in biomaterial applications is type I collagen, which is a “rope-forming” collagen and is ubiquitous in the body, including skin and bone. Collagen can be resorbed into the body, is non-toxic, produces only a minimal immune response (even between different species), and is useful for attachment and biological interaction with cells. Collagen may also be processed into a variety of formats, including porous sponges, gels, and sheets, and can be crosslinked with chemicals to improve its strength or to alter its degradation rate.

Chitosan is derived from chitin, a type of polysaccharide (i.e., sugar) that is present in the hard exoskeletons of shellfish such as shrimp and crab. Chitin, in fact, is one of the most abundant polysaccharides found in nature, making chitosan a plentiful and relatively inexpensive product. Chitosan contains several desirable properties, including, but limited to, minimal foreign body reaction; mild processing conditions (synthetic polymers often need to be dissolved in harsh chemicals; chitosan will dissolve in water based on pH); controllable mechanical/biodegradation properties (e.g., scaffold porosity or polymer length); and availability of chemical side groups for attachment to other molecules. It can be combined with other materials in order to increase its strength and cell-attachment potential. Mixtures with synthetic polymers such as poly(vinyl alcohol) and poly(ethylene glycol), or natural polymers such as collagen, are available and have displayed improved performance over the behavior of either component alone.

According to some embodiments, the capture particle is a dextran bead coated with alginate. Dextrans are polysaccharides with molecular weights ≥1,000 Dalton. They have a linear backbone of α-linked glucopyranosyl repeating units. Dextrans are grouped into three (3) classes based on their structural features. Class 1 dextrans, which contain the α(1→6)-linked D-glucopyranosyl backbone modified with small side chains of D-glucose branches with α(1→2), α(1→3), and α(1→4)-linkage, vary in their molecular weight, spatial arrangement, type and degree of branching, and length of branch chains, depending on the microbial producing strains and cultivation conditions. Isomaltose and isomaltotriose are oligosaccharides with the class 1 dextran backbone structure. Class 2 dextrans (alternans) contain a backbone structure of alternating α(1→3) and α(1→6)-linked D-glucopyranosyl units with α(1→3)-linked branches. Class 3 dextrans (mutans) have a backbone structure of consecutive α(1→3)-linked D-glucopyranosyl units with α(1→6)-linked branches. One and two-dimensional NMR spectroscopy techniques have been utilized for the structural analysis of dextrans. The physical and chemical properties of purified dextrans vary depending on the microbial strains from which they are produced and by the production method. Dextrans have high water solubility and the solutions behave as Newtonian fluids. Solution viscosity depends on concentration, temperature, and molecular weight, which have a characteristic distribution. The hydroxyl groups present in dextran offer many sites for derivatization, and these functionalized glycoconjugates represent a largely unexplored class of biocompatible and environmentally safe compounds.

According to some embodiments, the capture particle is lysed with a lysing agent. According to some embodiments, the lysing agent is a chelating agent. According to some embodiments, the chelating agent is a calcium (Ca²⁺) chelating agent. Calcium chelating agents include, but are not limited to, ethylenediaminetetraacetic acid (EDTA); ethylene glycol tetraacetic acid (EGTA); 1,2-bis(o-aminophenoxy)ethane-N,N,N′,N′-tetraacetic acid (BAPTA); deferoxamine mesylate, iron chelator IV, 21H7; and N,N,N′,N′-tetrakis(2-pyridylmethy)ethane-1,2-diamine (TPEN).

According to some embodiments, the capture particle is coated with a conjugate. Non-limiting examples of conjugates include heavy chain conjugates, light chain conjugates, avidin and streptavidin. Examples of heavy chain conjugates include, but are not limited to, Protein A, recombinant Protein A, Protein G and recombinant Protein G. Examples of light chain conjugates include, but are not limited to, Protein L and recombinant Protein L.

Protein A is derived from Staphylococcus aureus. Protein G is derived from a Streptococcus species. Both have binding sites for the Fc portion of mammalian IgG. The affinity of these proteins for IgG varies with the animal species. Protein G has a higher affinity for rat, goat, sheep, and bovine IgG, as well as for mouse IgG1 and human IgG3. Protein A has a higher affinity for cat and guinea pig IgG. Native Protein G contains binding sites for albumin, the Fab region of Igs, and membrane binding regions, which can lead to nonspecific interactions. Recombinant Protein G has been engineered to eliminate the albumin binding region, and recombinant Protein G′ is a truncated protein which lacks the albumin, Fab, and membrane binding sites while retaining the Fc binding site, making it more specific for IgG than the native form. Neither Protein A nor Protein G is recommended for detection of IgA or IgM, for detection of Fab fragments, or for detection of avian IgG. When bound to a resin such as alginate, agarose or Sepharose®, Protein A and Protein G can be used as affinity adsorbents to purify immunoglobulins and immunoglobulin subtypes from serum, hybridoma ascites fluids, tissue culture supernatants, and other biological fluids. These reagents are also commonly used to capture immune complexes generated in immunoprecipitation experiments.

Protein L is derived from Peptostreptococcus magnus. It has an affinity for kappa light chains from various species and will detect monoclonal or polyclonal IgG, IgA, and IgM as well as Fab, F(ab′)₂, and recombinant single-chain Fv (scFv) fragments that contain kappa light chains. It will also bind chicken IgG. Species such as bovine, goat, sheep, and horse, whose Igs contain almost exclusively lambda chains, will not bind well, if at all, to Protein L. Protein L is used as a general reagent for binding primary mammalian or avian antibodies or surface Igs of all classes. It is useful for detection of Fab, F(ab′)₂ fragments, recombinant scFv fragments, Igs bound to Fc receptors, or for detection of monoclonal antibodies in the presence of bovine Igs bearing kappa light chains.

Avidin, an egg-white protein, is a highly cationic 66,000-dalton glycoprotein with an isoelectric point of about 10.5. Its bacterial counterpart, streptavidin, is a non-glycosylated 52,800-dalton protein with a near-neutral isoelectric point. Streptavidin contains the tripeptide sequence Arg-Tyr-Asp (RYD) that apparently mimics the Arg-Gly-Asp (RGD) binding sequence of fibronectin, a component of the extracellular matrix that specifically promotes cellular adhesion. This universal recognition sequence binds integrins and related cell-surface molecules. Each avidin and streptavidin protein binds four (4) biotin molecules with high affinity (K_{d} of 10⁻¹⁴ to 10⁻¹⁵ mol/L) and selectivity. Biotin, also known as vitamin B₇, vitamin H or coenzyme R, is a water-soluble B-vitamin composed of a tetrahydroimidizalone ring fused with a tetrahydrothiophene ring. Because both avidin and streptavidin bind biotin with a high affinity and selectivity, proteins linked to biotin, or “biotinylated”, can be, for example, isolated from a sample or conjugated to an avidin/streptavidin coated surface.

According to some embodiments, the described invention provides antibodies that bind cell-surface markers. According to some embodiments, the antibodies are full-length. According to some embodiments, the antibodies are fragments. According to some embodiments, the fragment comprises only an antigen-binding portion. According to another embodiment, the antigen binding portion comprises a light chain variable region (V_{L}) and a heavy chain variable region (V_{H}). According to some embodiments, the antibodies are high-affinity antibodies. According to some embodiments, the antibodies are low-affinity antibodies.

Antibodies of the described invention include, but are not limited to, monoclonal antibodies, polyclonal antibodies and synthetic antibody mimics (SyAMs). Monoclonal antibodies include, but are not limited to, synthetic antibodies and engineered antibodies. Synthetic antibodies include, but are not limited to, recombinant antibodies. Recombinant antibodies include, but are not limited to, single-chain variable fragment (scFv) antibodies, nucleic acid aptamers and non-immunoglobulin protein scaffolds. Engineered antibodies include, but are not limited to, chimeric antibodies and humanized antibodies.

Monoclonal antibodies are a homogenous population of antibodies that recognize a single, specific eptitope of an antigen of interest. Monoclonal antibodies are produced in cell culture by hybridoma cells, which are the result of a fusion between myeloma cells and spleen cells from a mouse that has been immunized with a desired antigen or from myeloma cells and B-cells from a rabbit that has been immunized with the desired antigen.

Recombinant antibodies are antibodies that are produced by an in vitro expression system (i.e., not produced by immunizing an animal with a desired antigen). For example, the nucleic acid encoding a full-length antibody or V_{H} and V_{L} antigen binding domains may be inserted into a replicable vector for cloning (amplification of the DNA) or for expression. Various vectors are publicly available. The vector may, for example, be in the form of a plasmid, cosmid, viral particle, or phage. For example, plasmid vectors include, but are not limited to, pET-26+ and pCMV6-AC. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques.

By way of non-limiting example, expression and cloning vectors may contain a promoter operably linked to an antibody-encoding nucleic acid sequence to direct mRNA synthesis. Promoters recognized by a variety of potential host cells are well known. Promoters suitable for use with prokaryotic hosts include the beta-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Promoters for use in bacterial systems also can contain a Shine-Dalgarno (S.D.) sequence operably linked to DNA encoding antibodies.

Both expression and cloning vectors can contain a nucleic acid sequence that enables the vector to replicate in one or more selected host cells. Such sequences are known for a variety of bacteria, yeast, and viruses.

Expression and cloning vectors will typically contain a selection gene, also termed a selectable marker. Typical selection genes encode proteins that (a) confer resistance to antibiotics or other toxins (e.g., ampicillin, neomycin, methotrexate, or tetracycline), (b) complement auxotrophic deficiencies, or (c) supply critical nutrients not available from complex media, (e.g., the gene encoding D-alanine racemase for Bacilli). Examples of selectable markers for mammalian cells include, but are not limited to, those that enable the identification of cells competent to take up an antibody-encoding nucleic acid, such as DHFR or thymidine kinase. An exemplary host cell when wild-type DHFR is employed is the CHO cell line deficient in DHFR activity, prepared and propagated as described by Urlaub et al., Proc. Natl. Acad. Sci. USA, 77:4216 (1980). An exemplary selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)).

Host cells are transfected or transformed with expression or cloning vectors described herein for antibody production and cultured in conventional nutrient media modified as appropriate for inducing promoters, selecting transformants, or amplifying the genes encoding the desired sequences. In general, principles, protocols, and practical techniques for maximizing the productivity of cell cultures can be found in Mammalian Cell Biotechnology: a Practical Approach, M. Butler, ed. (IRL Press, 1991).

Methods of eukaryotic cell transfection and prokaryotic cell transformation include, for example, CaCl₂, Ca₂PO₄, liposome-mediated and electroporation. Depending on the host cell used, transformation is performed using standard techniques appropriate to such cells. The calcium treatment employing calcium chloride, as described in Sambrook et al., or electroporation is generally used for prokaryotes. For mammalian cells, the calcium phosphate precipitation method of Graham and van der Eb, Virology, 52:456-457 (1978) can be employed. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, See Keown et al., Methods in Enzymology, 185:527-537 (1990) and Mansour et al., Nature, 336:348-352 (1988).

Suitable host cells for cloning and expressing DNA, and for producing recombinant antibodies include, but are not limited to, Gram-negative bacteria, Gram-positive bacteria, yeasts, fungi, protozoa, insect cells, mammalian cells and transgenic plants.

Escherichia coli has been the most important Gram-negative production system for recombinant proteins reaching volumetric yields in the gram per liter scale for extracellular production. However, cell wall-less L-forms of the Gram-negative bacterium Proteus mirabilis and Pseudomonas putidas have been used for the production of mini antibodies and scFv.

Gram-positive bacteria directly secrete proteins into the medium due to the lack of an outer membrane which facilitate production of antibody fragments. The Gram-positive bacteria Bacillus brevis, Bacillus subtilis, and Bacillus megaterium have been successfully used for the production of different antibody fragments. In addition, B. megaterium does not produce alkaline proteases and provides high stability of plasmid vectors during growth allowing stable transgene expression during long term cultivation in bioreactors. Lactobacilli have also been tested for antibody production and are “generally regarded as safe” (GRAS) microorganisms. To date, two lactobacillus strains have been used for the production of scFvs, Lactobacillus zeae/casei, and Lactobacillus paracasei.

Yeast combine short generation time and ease of genetic manipulation with the robustness and simple medium requirements of unicellular microbial hosts. Pichia pastoris is an exemplary yeast strain used for recombinant antibody production. Other yeast like Saccharomyces cerevisiae, Hansenula polymorpha, Schizosaccharomyces pombe, Schwanniomyces occidentalis, Kluyveromyces lactis, and Yarrowia lipolytica have also been described for protein production.

Mammalian cells offer advanced mammalian folding, and a secretion and post-translational apparatus that is capable of producing antibodies indistinguishable from those in the human body with least concerns for immunogenic modifications. They are also highly efficient for secretion of large and complex IgGs and, in combination with the folding and post-translational control, high product quality can be achieved which reduces efforts and costs in the subsequent and more expensive downstream processing steps. The risks of contamination by pathogens or bovine spongiform encephalopathy (TSE/BSE) agents have been eliminated by well-documented Good Manufacturing Practice (GMP) compliant designer cell substrates and chemical defined media without the need of supplementing animal serum components. Mammalian cell culture technology can reach production levels of approximately 5 g/L IgGs in Chinese hamster ovary (CHO) cells. Industrial IgG production levels often exceed 12 g/L as the result of a steadily ongoing progress in mammalian cell culture technology, mainly due to improved high producer cell lines, optimized production media, and prolonged production processes at high-cell densities. Producer cell lines have also been genetically engineered regarding product homogeneity, improved metabolism, reduced apoptosis, and inducible cell cycle arrest which allow prolonged production times for almost 3 weeks at high-cell viability and cell densities.

Chinese hamster ovary (CHO) cells are the most common cells applied in the commercial production of biopharmaceuticals. This cell line isolated in the 1950s gave rise to a range of genetically different progeny, such as K1-, DukX B11-, DG44-cell lines and others which differ in protein product quality and achievable yield. In addition, Per.C6 cells, mouse myeloma NS0 cells, baby hamster kidney (BHK) cells and the human embryonic kidney cell line HEK293 have also been used for recombinant protein production. Although glycosylation patterns of mammalian glycoproteins are very similar to that in humans, even small differences can influence pharmacokinetics and effector functions of antibodies. Alternative designer cell lines with improved glycosylation patterns have been generated, for example human neuronal precursor cell line AGE1.HN (Probiogen, Berlin, Germany) supporting specific and complex glycostructures for the production of antibodies which require specific post-translational modifications or suffer from instability or susceptibility for proteolysis. CHO cell variant Lec13 (Glycotope) also produces human IgG with N-Linked glycans lacking fucose which improves on Fc-gammaRIII binding and antibody-dependent cell-mediated cytotoxicity.

A single-chain fragment variable fragment (scFv) antibody consists of variable regions of heavy (V_{H}) and light (V_{L}) chains, which are joined together by a flexible peptide linker. To create a scFv gene, mRNA is isolated from hybridoma (or also from the spleen, lymph cells, and bone morrow) cells from an immunized animal (e.g., mouse), followed by reverse transcription into cDNA to serve as a template for antibody gene amplification (PCR). Once the DNA fragments encoding V_{H} and V_{L} segments are obtained (by amplification and mutagenesis of germline V_{H} and V_{L} genes, as described above), these DNA fragments can be further manipulated by standard recombinant DNA techniques, for example to convert the variable region genes to full-length antibody chain genes, to Fab fragment genes or to a scFv gene. The isolated DNA encoding the V_{H} region can be converted to a full-length heavy chain gene by operatively linking the V_{H}-encoding DNA to another DNA molecule encoding heavy chain constant regions (CH1, CH2 and CH3). The sequences of human heavy chain constant region genes are known, and DNA fragments encompassing these regions can be obtained by standard PCR amplification. The heavy chain constant region can be an IgG1, IgG2, IgG3, IgG4, IgA, IgE, IgM or IgD constant region. For an Fab fragment heavy chain gene, the V_{H}-encoding DNA can be operatively linked to another DNA molecule encoding only the heavy chain CH1 constant region. The isolated DNA encoding the V_{L} region can be converted to a full-length light chain gene (as well as a Fab light chain gene) by operatively linking the V_{L}-encoding DNA to another DNA molecule encoding the light chain constant region, CL. The sequences of human light chain constant region genes are known, and DNA fragments encompassing these regions can be obtained by standard PCR amplification. The light chain constant region can be a kappa or lambda constant region. The V_{H}- and V_{L}-encoding DNA fragments are operatively linked to another fragment encoding a flexible linker, e.g., encoding the amino acid sequence (Gly₄ Ser)₃, or (Gly₄Ser)₄ such that the V_{H} and V_{L} sequences can be expressed as a contiguous single-chain protein, with the V_{L} and V_{H} regions joined by the flexible linker. The term “operatively linked”, as used in this context, is defined to mean that the two DNA fragments are joined such that the amino acid sequences encoded by the two DNA fragments remain in-frame.

Nucleic acid aptamers are small RNA/DNA molecules that can form secondary and tertiary structures capable of specifically binding proteins or other cellular targets. They are essentially a chemical equivalent of antibodies and they have the advantage of being highly specific, relatively small in size, and non-immunogenic. Aptamers can be generated, for example, by a process called systematic evolution of ligands by exponential enrichment (SELEX). SELEX involves the progressive selection, from a large combinatorial oligonucleotide library, of DNA and/or RNA ligands with variable DNA-binding and/or RNA-binding affinities and specificities by repeated rounds of partition and amplification.

Non-immunoglobulin (non-Ig) protein scaffolds are small, single-domain proteins that require no post-translational modification, often lack disulfide bonds, and can undergo multimerization. These scaffolds can be equipped with novel binding sites by employing methods of combinatorial engineering, such as site-directed random mutagenesis in combination with phage display or other molecular selection techniques. They are derived from robust and small soluble monomeric proteins (e.g., Kunitz inhibitors or the lipocalins) or from stably folded extra-membrane domains of cell surface receptors (e.g., protein A, fibronectin or the ankyrin repeat). Compared with antibodies or their recombinant fragments, these protein scaffolds often provide advantages including, but not limited to, elevated stability and high production yield in microbial expression systems.

Chimeric monoclonal antibodies are therapeutic biological agents containing murine or other non-human variable regions, which target the antigen of interest, and human Fc Ig components, which reduce the immunogenicity of the antibody. These antibodies are produced in mammalian expression systems using specially designed vectors and selectable markers. For example, an antibody (mouse or other non-human) variable region can be subcloned into a vector for construction of a chimeric antibody with a human IgG backbone (IgG1, 2, 3, or 4). Once the sequence is confirmed, the expression vector can be transfected into a mammalian cell, such as Chinese hamster ovary (CHO-S) using an Amaxa Nucleofector II. The supernatant of transfectant pools can be purified by Protein A chromatography.

Humanized monoclonal antibodies typically retain only the hypervariable regions or complementary determining regions (CDRs) of a murine (or other non-human) antibody while the remainder of the antibody is human. Humanized antibodies typically contain about 5% to about 10% murine (or other non-human) composition. Humanized monoclonal antibodies can be synthesized by grafting murine CDRs to a human antibody. Using recombinant DNA technology, human immunoglobulin light and heavy chain genes can be amplified by polymerase chain reaction (PCR). The resulting human lymphoid cDNA library can be used as a template for in vitro synthesis of the entire antibody, except for the CDRs. Murine (or other non-human) CDRs are cloned and grown in parallel. The respective genes can then be spliced into vector DNA and incorporated into a host cell (e.g., bacteria) for growth. To streamline the process, often both human cDNA and murine (or other non-human) cDNA containing vectors can be incorporated into the same host cell (co-transfection) and an intact humanized monoclonal antibody can be produced.

Addition of isopropyl-beta-D-thiogalactopyranoside (IPTG) to bacterial cultures can be used to induce expression of plasmid-based genes for the production of recombinant peptides under the control of the lac promoter. IPTG binds to the lac repressor in Escherichia coli, thereby preventing binding of the repressor protein to DNA and blocking gene transcription.

To express the antibodies, or antibody portions, DNAs encoding partial or full-length light and heavy chains are inserted into expression vectors such that the genes are operatively linked to transcriptional and translational control sequences. In this context, the term “operatively linked” is defined to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion, the expression vector may already carry antibody constant region sequences. The recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

After expression, recombinant antibody may be recovered from culture medium or from host cell lysates. If membrane-bound, it can be released from the membrane using a suitable detergent solution (e.g. Triton-X 100) or by enzymatic cleavage. Cells employed in expression of antibody can be disrupted by various physical or chemical means, such as freeze-thaw cycling, sonication, mechanical disruption, or cell lysing agents.

It may be desirable to isolate or purify antibody from recombinant cell proteins or polypeptides. Exemplary isolation and purification procedures include: size-exclusion chromatography (SEC), ammonium sulfate precipitation, ion exchange chromatography, immobilized metal chelate chromatography, thiophilic adsorption, melon gel chromatography, protein A, protein G, protein L and antigen-specific affinity purification. Various methods of protein purification may be employed (See, e.g., Deutscher, Methods in Enzymology, 182 (1990); Scopes, Protein Purification: Principles and Practice, Springer-Verlag, New York (1982)). The purification step(s) selected will depend, for example, on the nature of the production process used and the particular peptide produced.

Polyclonal antibodies are secreted by different B-cell lineages and are thus a collection of immunoglobulin molecules that react against different (multiple) epitopes of a specific antigen. These antibodies are generated by injecting an animal with an antigen. Animals suitable for polyclonal antibody generation include, but are not limited to, rabbit, mouse, rat, hamster, guinea pig, goat, sheep and chicken. Injections can be performed every 4-6 weeks. Animals can be bled 7-10 days after each injection. The quality and quantity of antibodies in serum (i.e., of the bleeds) can be monitored by an immunological assay such as enzyme-linked immunosorbent assay (ELISA). Antibody titer can be defined as the dilution yielding half maximal absorbance in the assay. Antibodies may be purified (i.e., separated from other serum proteins), for example, by Protein A affinity chromatography.

Synthetic antibody mimics (SyAMs), synthetic molecules that possess both the targeting and effector-cell-activating functions of antibodies, while being less than 1/20th (5%) of their molecular weight, are synthesized with an antigen binding domain and an Fc Gamma Receptor binding domain separated by structural peptides. SyAMs can be produced by molecular imprinting. Molecular imprinting is a technique used to create template-shaped cavities in polymer matrices with memory of the template molecules to be used in molecular recognition. The technique is based on enzyme-substrate recognition, also known as the “lock and key” model.

According to some embodiments, the antibodies are human antibodies.

According to some embodiments, the described invention employs antibodies conjugated to capture particles, e.g., beads. Methods for conjugating antibodies are known and include, without limitation, affinity immobilization, amine-reactive immobilization, sulfhydryl-reactive immobilization, carbonyl-reactive immobilization, carboxyl-reactive immobilization and active hydrogen immobilization.

Affinity immobilization includes, but is not limited to, Protein A coated beads, Protein G coated beads, Protein L coated beads and avidin/streptavidin coated beads—biotin labelled antibody.

Amine-reactive immobilization includes, but is not limited to, cyanogen bromide (CNBr) activation, N-hydroxysuccinimide (NHS) ester activation, aldehyde activation, azlactone activation and carbonyl diimidazole (CDI) activation. Amine-reactive immobilization methods target the amine group (—NH₂) of a protein molecule. This group exists at the N-terminus of each polypeptide chain (called the alpha-amine) and in the side chain of lysine (Lys, K) residues (called the epsilon-amine). Because of their positive charge at physiologic conditions, primary amines are usually outward-facing (i.e, on the outer surface) of proteins. Thus, they are usually accessible for conjugation without denaturing the protein structure.

Sulfhydryl-reactive immobilization includes, but is not limited to, maleimide activation, iodoacetyl activation and pyridyl disulfide activation. Sulfhydryl-reactive immobilization uses the thiol group of a protein molecule to direct coupling reactions away from active centers or binding sites on certain protein molecules. Sulfhydryls (—SH) exists in the side chain of cysteine (Cys, C). As part of a protein's secondary or tertiary structure, cysteines can be joined together between their side chains via disulfide bonds (—S—S—). These must be reduced to sulfhydryls to make them available for immobilization. Sulfhydryl groups typically are present in fewer numbers than primary amines and, therefore, enable more selective immobilization of proteins and peptides. Sulfhydryls for conjugation can be added to peptide ligands at the time of peptide synthesis by adding a cysteine residue at one end of the molecule. This ensures that every peptide molecule will be oriented on a support (e.g., bead) in the same way after immobilization. Thiol groups (sulfhydryls) can be indigenous within a protein molecule or they may be added through the reduction of disulfides or through the use of various thiolation reagents.

Carbonyl-reactive immobilization includes, but is not limited to, hydrazide activation. Carbonyl-reactive immobilization involves coupling through carbonyl groups. Most biological molecules do not contain carbonyl ketones or aldehydes in their native state. However, such groups can be created on proteins in order to form a site for immobilization that directs covalent coupling away from active centers or binding sites. Glycoconjugates, such as glycoproteins or glycolipids, contain sugar residues that have hydroxyls on adjacent carbon atoms. These cis-diols can be oxidized with sodium periodate to create aldehydes as sites for covalent immobilization.

Carboxyl-reactive immobilization includes, but is not limited to, carbodiimide-mediated immobilization. A non-limiting example of a carbodiimide is 1-ethyl-3-(3-dimethylaminopropyl) carbodiimide hydrochloride (EDC). Carboxyl-reactive immobilization methods target the carboxyl group (—COOH) of a protein molecule. Peptides and proteins contain carboxyls (—COOH) at the C-terminus of each polypeptide chain and in the side chains of aspartic acid (Asp, D) and glutamic acid (Glu, E). Like primary amines, carboxyls are usually on the surface of protein structure. Carboxylic acids may be used to immobilize biological molecules through the use of a carbodiimide-mediated reaction. Although no activated support contains a reactive group that is spontaneously reactive with carboxylates, chromatography supports containing amines (or hydrazides) can be used to form amide bonds with carboxylates that have been activated with the water-soluble carbodiimide crosslinker EDC.

Active hydrogen immobilization methods involve coupling through reactive hydrogens by condensing these hydrogens with formaldehyde and an amine using a reaction called the Mannich reaction. The Mannich reaction consists of the condensation of formaldehyde (or another aldehyde) with ammonia and another compound containing an active hydrogen. Instead of using ammonia, this reaction can be performed with primary or secondary amines or even with amides. Immobilization occurs when a diaminodipropylamine (DADPA) resin is used as the primary amine for this reaction.

According to some embodiments, the closed, automated system of the described invention comprises counterflow centrifugation (centrifugal elutriation).

Counterflow centrifugation separates particles (e.g., cells) based on density and size. During counterflow centrifugation, a sample of heterogeneous cells is passed into a chamber (e.g., triangular in shape) embedded in a centrifuge rotor/chamber while the rotor is in motion (i.e., spinning). Centrifugal force pushes cells away from the wider end of the chamber, while a counterflow produces an opposing force toward the smaller end. Sedimentation can occur toward an inlet located at the wider end of the chamber.

In counterflow centrifugation, particle sedimentation in a radial direction is balanced by the velocity of fluid flowing in the opposite direction. The flow velocity (V) at any point is equal to the flow rate (F), divided by the cross-sectional area at that point (A), V=F/A. Since the flow rate is the same at every point in the chamber, only changes in the cross-sectional area produce changes in the flow velocity. Thus, at chamber positions with small cross-sectional area (for example, at maximum radial position or rₘₐₓ), flow velocity is highest, and vice versa. Through chamber design, a velocity gradient is formed in the elutriation chamber using constant flow. A gradient in centrifugal force is introduced along the radial direction of the chamber, as centrifugal force is related to the rotor radius or distance from the center of the rotor. At rₘₐₓ, the force of centrifugation is greatest. However, the flow velocity is also greatest at this point as the cross-sectional area of the chamber is smallest. Closer to the center of the rotor, both the centrifugal force and flow velocity decrease as r (radial position) is shortened and A (cross-sectional area) increases across the chamber, respectively. When the opposing forces are equal, the system is said to be in equilibrium; i.e., in a state where smaller cells stay at rest near the elutriation boundary (i.e., closest to the center of the rotor) and larger cells remain stationary near the flow inlet (rₘₐₓ). Thus, separations are the result of cells of different sedimentation velocities being in equilibrium at different radial positions in the chamber. When the flow rate is increased (or the speed is decreased), cells that were in equilibrium near the elutriation boundary (i.e., smaller cells) are washed out of the chamber first and the distribution of cells at equilibrium shifts toward the center of rotation.

According to some embodiments, the described invention provides a centrifuge rotor with a vertical axis of rotation. According to some embodiments, the described invention provides a centrifuge rotor with a horizontal axis of rotation.

According to some embodiments, the selected/isolated cells are collected in a final product bag by decreasing rotor speed; by increasing flow rate of counterflow; or a combination thereof.

According to some embodiments, the capture particle is of a separable size, density, buoyancy or combination thereof. According to some embodiments, the capture particle recognizes and binds a target cell within a heterogeneous cell population. According to some embodiments, the capture particle bound to a target cell within a heterogeneous cell population is effective to change at least one of size, density and buoyancy of the target cell. According to some embodiments, the target cell bound to the capture particle is selected/isolated based on size, density, buoyancy or a combination thereof. According to some embodiments, the target cell bound to the capture particle is selected/isolated by counterflow centrifugation.

According to some embodiments, the described invention provides a method for labelling cells with a capture particle comprising a magnetic component. According to some embodiments, the magnetic component is a magnetic particle. According to some embodiments, the magnetic particle is a microparticle. According to some embodiments, the magnetic particle is a nanoparticle. According to some embodiments, the magnetic particle comprises iron. According to some embodiments, the magnetic particle comprises iron dextran. According to some embodiments, the capture particle comprises an iron dextran particle to which an antibody has been coupled.

According to some embodiments, the method comprises incubating a heterogeneous cell population with a capture particle to label a targeted population of cells, According to some embodiments, the capture particle comprises a magnetic component. According to some embodiments, binding of the capture particle comprising the magnetic component to the target cell within the heterogeneous cell population is effective to change at least one of size, density and buoyancy of the target cell relative to the unlabeled cells in the heterogeneous cell population. According to some embodiments, the target cell bound to the capture particle comprising the magnetic component is selected/isolated based on its size, density, buoyancy or a combination thereof. According to some embodiments, the target cell bound to the capture particle comprising the magnetic component is selected/isolated by counterflow centrifugation.

According to some embodiments, the described invention provides a method for labelling cells with a capture particle comprising a magnetic component and separating/isolating the labelled cells using an automated, closed system (100).

According to some embodiments, the capture particle comprising a magnetic component recognizes and binds a target cell within the heterogeneous cell population. According to some embodiments, the target cell bound to the magnetic capture particle is separated/isolated magnetically by application of a magnetic field. According to some embodiments, the target cell bound to the magnetic capture particle is separated/isolated by counterflow centrifugation.

According to some embodiments, the method comprises mixing a heterogeneous cell population and capture particles comprising a magnetic component. According to some embodiments, the capture particles are antibodies coupled to iron dextran nanoparticles. According to some embodiments, the antibodies recognize a specific cell surface marker and bind (i.e. label) those cells within the heterogeneous cell population that contain the specific cell surface marker (i.e. target cells). According to some embodiments, the target cells are labelled before undergoing counterflow centrifugation. According to some embodiments, the capture particle bound to a target cell within a heterogeneous cell population is effective to change at least one of size, density and buoyancy of the target cell. According to some embodiments, the target cell bound to the capture particle is selected/isolated based on size, density, buoyancy or a combination thereof. According to some embodiments, the target cell bound to the capture particle is selected/isolated by counterflow centrifugation. According to some embodiments, the labeled target cell is separated from unbound nontargeted cells using a magnetic field. According to some embodiments, excess capture particles can be collected using a magnet. According to some embodiments, after separation/isolation, the target cells can be washed. According to some embodiments, separated/isolated target cells can be eluted from capture particles by use of an elution buffer, e.g., 100 mM citric acid, pH 3.0.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Selection/Isolation of Regulatory T-Cells (T_{reg} Cells) from a Heterogeneous Population of Cells

Regulatory T-cells (Treg cells) are selected/isolated from a heterogeneous population of leukocytes using the system and method of the described invention.

A heterogeneous population of leukocytes is prepared from whole blood using apheresis. Briefly, whole blood is introduced into a spinning centrifuge chamber and separates into plasma, platelet rich plasma, leukocytes and red blood cells by gravity along the wall of the chamber. Leukocytes are removed by moving an aspiration device to the level of separated leukocytes and suspended in a physiological medium.

Capture particles are prepared by immobilizing an anti-human CD4 antibody (sc-514571, Santa Cruz Biotechnology, Dallas, Tex.), anti-human CD25 antibody (MAB623, R&D Systems, Minneapolis, Minn.), anti-human CD127 antibody (306-IR, R&D Systems), anti-human FOXP3 antibody (ab54501, abcam, Cambridge, Mass.) or a combination of these antibodies on an alginate microsphere. For example, an antibody or antibody combination is immobilized on the porous network of the alginate microsphere during external cross-linking of the alginate with divalent or polyvalent cations (e.g., Ca²⁺ from CaCl₂). Antibody or antibody combinations are added to a vial of sodium (Na)-alginate-tris buffered saline (TBS) solution containing CaCl₂ and the vial is placed on a reciprocating shaker (Thermo Scientific) with gentle motion at 10 rpm. The alginate gel microspheres are then centrifuged at 800 g for 5 min to collect the alginate microspheres with the immobilized antibodies.

Using the system of the described invention, the heterogeneous population of leukocytes suspended in the physiological medium is mixed with the capture particles in a chamber embedded in a centrifuge rotor while the rotor is in motion and a counterflow in the chamber produces an opposing force within the chamber. Capture particles are bound to T_{reg} cells in the chamber embedded in the centrifuge rotor while the rotor is in motion and the counterflow produces an opposing force within the chamber. Wash buffer (e.g., physiological medium) is then passed through the chamber embedded in the centrifuge rotor while the rotor is in motion and the counterflow produces an opposing force within the chamber in order to remove unbound cells and unbound capture particles from the chamber. Next, a lysing agent (e.g., EDTA) is added to the chamber embedded in the centrifuge rotor while the rotor is in motion and the counterflow produces an opposing force within the chamber in order to lyse the alginate beads. Wash buffer is passed through the chamber embedded in the centrifuge rotor while the rotor is in motion and the counterflow produces an opposing force within the chamber in order to remove the lysing agent and the lysed bead from the chamber. Next, T_{reg} cells bound to antibody are collected and subsequently are dissociated from antibody using a dissociation solution (e.g., 100 mM citric acid, pH 3.0).

### Example 2. Selection/Isolation of Hematopoietic Stem Cells from a Heterogeneous Population of Cells

Hematopoietic stem cells are selected/isolated from a heterogeneous population of leukocytes using the system and method of the described invention.

A heterogeneous population of leukocytes is prepared from whole blood using apheresis. Briefly, whole blood is introduced into a spinning centrifuge chamber and separates into plasma, platelet rich plasma, leukocytes and red blood cells by gravity along the wall of the chamber. Leukocytes are removed by moving an aspiration device to the level of separated leukocytes and suspended in a physiological medium.

Capture particles are prepared by immobilizing an anti-human CD34 antibody (EPR2999, abcam, Cambridge, Mass.) on an alginate microsphere. For example, the antibody is immobilized on the porous network of the alginate microsphere during external cross-linking of the alginate with divalent or polyvalent cations (e.g., Ca²⁺ from CaCl₂). Antibody is added to a vial of sodium (Na)-alginate-tris buffered saline (TBS) solution containing CaCl₂ and the vial is placed on a reciprocating shaker (Thermo Scientific) with gentle motion at 10 rpm. The alginate gel microspheres are then centrifuged at 800 g for 5 min to collect the alginate microspheres with the immobilized anti-CD34 antibodies.

Using the system of the described invention, the heterogeneous population of leukocytes suspended in the physiological medium is mixed with the capture particles in a chamber embedded in a centrifuge rotor while the rotor is in motion and a counterflow in the chamber produces an opposing force within the chamber. Capture particles are bound to hematopoietic stem cells in the chamber embedded in the centrifuge rotor while the rotor is in motion and the counterflow produces an opposing force within the chamber. Wash buffer (e.g., physiological medium) is then passed through the chamber embedded in the centrifuge rotor while the rotor is in motion and the counterflow produces an opposing force within the chamber in order to remove unbound cells and unbound capture particles from the chamber. Next, a lysing agent (e.g., EDTA) is added to the chamber embedded in the centrifuge rotor while the rotor is in motion and the counterflow produces an opposing force within the chamber in order to lyse the alginate beads. Wash buffer is passed through the chamber embedded in the centrifuge rotor while the rotor is in motion and the counterflow produces an opposing force within the chamber in order to remove the lysing agent and the lysed bead from the chamber. Next, hematopoietic stem cells bound to antibody are collected and subsequently are dissociated from antibody using a dissociation solution (e.g., 100 mM citric acid, pH 3.0).

### Example 3. Transduction of Cells

The term “transfection” as used herein refers to experimental introduction of foreign DNA into cells in culture, usually followed by expression of genes in the introduced DNA. Virus-mediated transfection or transduction is a process whereby transfer of genetic material (and its phenotypic expression) from one cell to another occurs by viral infection. Virus-mediated transfection is highly efficient and it is easy to achieve sustainable transgene expression in vivo owing to the viral nature of DNA integration into the host genome, and integrated DNA expression in the host.

A standard protocol for transfecting mammalian cells is as follows. 2×10⁶ human embryonic kidney cells (293T cells; ATCC, Manassas, Va.) are seeded on a 100-cm tissue culture dish (Corning, Inc., Corning, N.Y.) and incubated until the cells are approximately 70% confluent (roughly 1-2 days). A viral vector (meaning an agent that can carry DNA into a cell or organism) is prepared by adding an 8:1 ratio of a packaging plasmid (meaning a small circular extrachromosomal DNA molecule capable of autonomous replication in a cell (e.g., pUMVC3 (Aldevron, Fargo, N. Dak.) or pLenti-C-Myc-DDK-IRES-Puro (Origene, Rockville, Md.)) containing genetic material of interest to a packaging or envelop plasmid (e.g., pCMV-VSV-G (Cell Biolabs, Inc., San Diego, Calif.)) for a total of 1 μg to a polypropylene tube containing 94 μL of serum-free DMEM (Sigma-Aldrich, St. Louis, Mo.). Next, 6 μL of FuGENE® transfection reagent (Promega, Madison, Wis.) is added to the tube; the tube contents are mixed by pipetting and incubated at room temperature for 20-30 minutes. After incubation, transfection of 293T cells is performed by adding the viral vector mixture dropwise to the 293T cells and incubating the cells overnight. Virus-containing media is first collected 48 hours after transfection and subsequently every 12 hours for a total of 3 collections. The collected viral media is passed through a 0.45 μM low protein binding filter (EMD Millipore, Billerica, Mass.). Next, the viral vector is concentrated by transferring the filtered viral media to an Amicon filter (EMD Millipore, Billerica, Mass.) and centrifuging at 3,000 rpm for 10-20 minutes at 4° C.

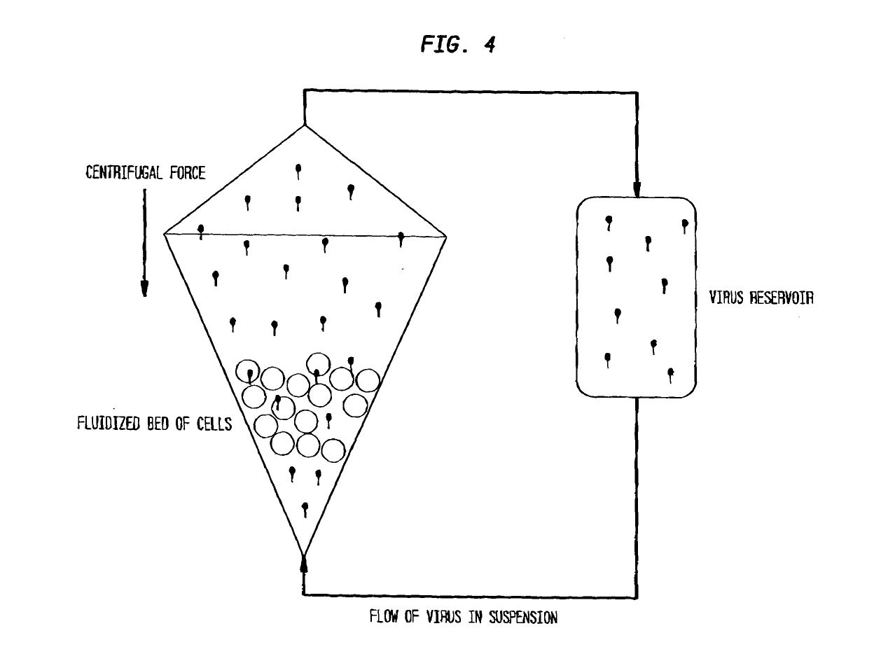

Mammalian cells can be transduced using the system and method of the described invention which is effective for transducing cells and does not require cell pelleting, which may be damaging to cells. Mammalian cells are captured within a fluidized bed within the chamber embedded in the centrifuge rotor/chamber (FIG. 4). Next, a transduction buffer comprising the concentrated viral vector that is packaged with the genetic material of interest is circulated (i.e., continually passed) around the cells. The circulation increases the probability of virus-cell interaction resulting in viral-mediated gene transfer (FIG. 4). Without being bound by theory, this process can result in improved mixing, thus providing higher transduction efficiency; and in shorter incubation times.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. An automated, closed system for selecting a target cell population comprising:
a. an input bag comprising a population of cells suspended in a physiological medium;
b. a chamber embedded in a centrifuge rotor, into which the population of cells is passed;
c. a capture particle injector comprising an agent adapted
a. to identify a subpopulation of the population of cells;
b. to select the subpopulation of the population of cells; and
c. to be released from the subpopulation of the population of cells after the selection;
d. an output bag comprising the released capture particle; the selected cells, or both; and
e. a buffer bag comprising a wash buffer.

2. The automated, closed system according to claim 1, wherein the capture particle injector comprises a capture particle adapted to recognize and bind to a cell surface marker on a surface of the subpopulation of the population of cells.

3. The automated, closed system according to claim 2, wherein the capture particle comprises a labeling agent that recognizes and binds to the cell surface marker.

4. The automated, closed system according to claim 2, further comprising a labelling bag comprising a cell not bound to the capture particle and the capture particle not bound to a cell.

5. The automated, closed system according to claim 2, further comprising a labelling bag comprising a cell bound to a capture particle and a capture particle bound to a cell.

6. The automated, closed system according to claim 1, wherein the agent is further conjugated to a bead.

7. The automated, closed system according to claim 1, wherein the population of cells is a homogeneous cell population.

8. The automated, closed system according to claim 1, wherein the population of cells is a heterogeneous cell population.

9. The automated, closed system according to claim 1, further comprising a pump.

10. The automated, closed system according to claim 1, wherein the chamber is triangular-shaped.

11. The automated, closed system according to claim 3, wherein the labeling agent adapted to recognize and bind to the cell-surface marker is an antibody.

12. The automated, closed system according to claim 1, wherein the wash buffer is selected from the group consisting of Tris-buffered saline (TBS), phosphate buffered saline (PBS), Tris-buffered saline-tween-20 (TBST), phosphate-buffered saline-tween-20 (PBST), triethanolamine in PBS and a physiological medium.

13. The automated, closed system according to claim 12, wherein the physiological medium is selected from the group consisting of basal medium eagle (BME), Dulbecco's phosphate buffered saline (DPBS), Dulbecco's modified eagle medium (DMEM), DMEM-F12 media, F-10 nutrient mixture, Glasgow modified minimum essential medium (GMEM), Iscove's modified Delbucco's medium (IMDM), Leibovitz's L-15 medium, McCoy's 5A medium, MCDB 153 medium, media 199, minimal essential medium (MEM), minimal essential media alpha (MEMA), RPMI 1640 medium, CliniMACS® buffer, Hanks balanced salt saoltion (HBSS), TexMACs™ medium, and Waymouth's MB 752/1 medium.

14. The automated, closed system according to claim 6, further comprising a lysing agent bag comprising a lysing agent that is effective to lyse the bead.

15. The automated, closed system according to claim 14, wherein the lysing agent bag comprises a calcium chelating agent.

16. The automated, closed system according to claim 15, wherein the calcium chelating agent is selected from the group consisting of ethylenediaminetetraacetic acid (EDTA); ethylene glycol tetraacetic acid (EGTA); 1,2-bis(o-aminophenoxy)ethane-N,N,N′,N′-tetraacetic acid (BAPTA); deferoxamine mesylate, iron chelator IV, 21H7; and N,N,N′,N′-tetrakis(2-pyridylmethy)ethane-1,2-diamine (TPEN).

17. The automated, closed system according to claim 15, wherein the calcium chelating agent is ethylenediaminetetraacetic acid (EDTA).

18. The automated, closed system according to claim 6, wherein the bead is comprised of a natural polymer.

19. The automated, closed system according to claim 18, wherein the natural polymer is selected from the group consisting of alginate, an alginate derivative, agarose, cross-linked agarose (Sepharose®), collagen and chitosan.

20. The automated, closed system according to claim 18, wherein the natural polymer is alginate.

21. The automated, closed system according to claim 6, wherein the bead comprises dextran coated with alginate.

22. The automated, closed system according to claim 6, wherein the bead is a microbead.

23. The automated, closed system according to claim 11, wherein the antibody is selected from the group consisting of a monoclonal antibody, a polyclonal antibody and a synthetic antibody mimic.

24. The automated, closed system according to claim 23, wherein the monoclonal antibody is selected from the group consisting of a synthetic antibody and an engineered antibody.

25. The automated, closed system according to claim 24, wherein the synthetic antibody is a recombinant antibody.

26. The automated, closed system according to claim 25, wherein the recombinant antibody is selected from the group consisting of a single-chain variable fragment (scFv) antibody, a nucleic acid aptamer and non-immunoglobulin protein scaffold.

27. The automated, closed system according to claim 24, wherein the engineered antibody is selected from the group consisting of a chimeric antibody and a humanized antibody.

28. A method for isolating a substantially pure population of cells from a heterogeneous cell suspension using the automated, closed system according to claim 1, comprising:
a. mixing a heterogeneous cell population with capture particles in a chamber embedded in a centrifuge rotor while the rotor is in motion and a counterflow in the chamber produces an opposing force within the chamber, wherein the capture particles comprise a bead conjugated to an agent that recognizes a specific cell surface marker;
b. binding cells to the capture particles in the chamber embedded in the centrifuge rotor while the rotor is in motion and the counterflow produces an opposing force within the chamber, wherein the cells bound to capture particles express the specific cell-surface marker recognized by the agent that recognizes the specific cell surface marker;
c. passing a wash buffer through the chamber embedded in the centrifuge rotor while the rotor is in motion and the counterflow produces an opposing force within the chamber, wherein the wash buffer removes unbound cells and unbound capture particles from the chamber;
d. collecting the cells bound to the agent that recognizes the specific cell surface marker, wherein the cells bound to the agent that recognizes the specific cell surface marker are enriched relative to the heterogeneous cell suspension; and
e. dissociating the cells in d. from the agent that recognizes the specific cell surface marker,
wherein the method is effective to:
(i) reduce the risk of contamination of the collected cells;
(ii) reduce damage to the collected cells;
(iii) maintain viability of the collected cells; or
(iv) a combination thereof.

29. The method according to claim 28, wherein the bead is comprised of a natural polymer.

30. The method according to claim 29, wherein the natural polymer is selected from the group consisting of alginate, an alginate derivative, agarose, cross-linked agarose (Sepharose®), collagen and chitosan.

31. The method according to claim 29, wherein the natural polymer is alginate.

32. The method according to claim 28, wherein the bead comprises dextran coated with alginate.

33. The method according to claim 28, wherein the bead is a microbead.

34. The method according to claim 28, wherein the agent that recognizes the specific cell surface marker is an antibody.

35. The method according to claim 34, wherein the antibody is selected from the group consisting of a monoclonal antibody, a polyclonal antibody, an engineered antibody, and a synthetic antibody mimic.

36. The method according to claim 35, wherein the synthetic antibody mimic is a recombinant antibody.

37. The method according to claim 36, wherein the recombinant antibody is selected from the group consisting of a single-chain variable fragment (scFv) antibody, a nucleic acid aptamer and a non-immunoglobulin protein scaffold.

38. The method according to claim 35, wherein the engineered antibody is selected from the group consisting of a chimeric antibody and a humanized antibody.

39. The method according to claim 28, wherein the wash buffer is selected from the group consisting of Tris-buffered saline (TBS), phosphate buffered saline (PBS), Tris-buffered saline-tween-20 (TBST), phosphate-buffered saline-tween-20 (PBST), triethanolamine in PBS and a physiological medium.

40. The method according to claim 39, wherein the physiological medium is selected from the group consisting of basal medium eagle (BME), Dulbecco's phosphate buffered saline (DPBS), Dulbecco's modified eagle medium (DMEM), DMEM-F12 media, F-10 nutrient mixture, Glasgow modified minimum essential medium (GMEM), Iscove's modified Delbucco's medium (IMDM), Leibovitz's L-15 medium, McCoy's 5A medium, MCDB 153 medium, media 199, minimal essential medium (MEM), minimal essential media alpha (MEMA), RPMI 1640 medium, CliniMACS® buffer, Hanks balanced salt saoltion (HBSS), TexMACs™ medium, and Waymouth's MB 752/1 medium.

41. The method according to claim 28, further comprising
adding a lysing agent to the chamber embedded in the centrifuge rotor while the rotor is in motion, the counterflow produces an opposing force within the chamber, wherein the lysing agent lyses the bead.

42. The method according to claim 41, further comprising
passing a wash buffer through the chamber embedded in the centrifuge rotor while the rotor is in motion, the counterflow producing an opposing force within the chamber, wherein the wash buffer removes the lysing agent and the lysed bead.

43. The method according to claim 41, wherein the lysing agent is a calcium chelating agent.

44. The method according to claim 43, wherein the calcium chelating agent is selected from the group consisting of ethylenediaminetetraacetic acid (EDTA); ethylene glycol tetraacetic acid (EGTA); 1,2-bis(o-aminophenoxy)ethane-N,N,N′,N′-tetraacetic acid (BAPTA); deferoxamine mesylate, iron chelator IV, 21H7; and N,N,N′,N′-tetrakis(2-pyridylmethy)ethane-1,2-diamine (TPEN).

45. The method according to claim 43, wherein the calcium chelating agent is ethylenediaminetetraacetic acid (EDTA).

46. The method according to claim 28, wherein the collecting in (d) is performed by stopping the motion of the centrifuge rotor, increasing rate of the counterflow or a combination thereof.

47. The method according to claim 28, wherein the contamination is selected from the group consisting of bacterial contamination, viral contamination, fungal contamination and cellular debris.

48. The method according to claim 28, wherein the damage is selected from the group consisting of cellular swelling, fat accumulation, metabolic failure, structural damage/deterioration and apoptosis.

49. The method according to claim 28, wherein the dissociating in (e) is performed with a dissociation solution.

50. The method according to claim 49, wherein the dissociation solution is selected from the group consisting of a pH solution, an ionic strength solution, a denaturing solution and an organic solution.

51. The method according to claim 50, wherein the pH solution is selected from the group consisting of 100 mM glycine-HCl, pH 2.5-3.0; 100 mM citric acid, pH 3.0; 50-100 mM trimethylamine or triethanolamine, pH 11.5; and 150 mM ammonium hydroxide, pH 10.5.

52. The method according to claim 50, wherein the ionic strength solution is selected from the group consisting of 3.5-4.0 M magnesium chloride, pH 7.0 in 10 mM Tris; 5 M lithium chloride in 10 mM phosphate buffer, pH 7.2; 2.5 M sodium iodide, pH 7.5; and 0.2-3.0 M sodium thiocyanate.

53. The method according to claim 50, wherein the denaturing solution is selected from the group consisting of 2-6 M guanidine-HCl; 2-8 M urea; 1% deoxycholate; and 1% sodium dodecyl sulfate (SDS).

54. The method according to claim 50, wherein the organic solution is selected from the group consisting of 10% dioxane and 50% ethylene glycol, pH 8-11.5.

55. The method according to claim 28, further comprising isolating the labeled targeted subpopulation of cells from the heterogeneous cell population based on size, density, buoyancy or a combination thereof of the labeled targeted subpopulation of cells, wherein
(a) the capture particle is effective to alter size, density, buoyancy or a combination thereof of the target cell, and
(b) binding of the capture particle comprising the agent that recognizes and binds specifically to the target subpopulation of cells within the heterogeneous cell population is effective to change at least one of size, density and buoyancy of each target cell relative to an unlabeled cell in the heterogeneous cell population.

56. A method for efficient viral-mediated gene transfer in mammalian cells comprising:
a. Providing a first input bag containing a mammalian cell population and a second input bag containing a transduction buffer comprising a concentrated viral vector that is packaged with genetic material foreign to the mammalian cell population;
b. Adding the first input bag containing the mammalian cell population and the transduction buffer comprising a concentrated viral vector that is packaged with genetic material foreign to the mammalian cell population to a chamber embedded in a centrifuge rotor while the rotor is in motion and a counterflow in the chamber produces an opposing force within the chamber;
c. Incubating the mammalian cell population with the concentrated viral vector packaged with genetic material foreign to the mammalian cell population by circulating the transduction buffer comprising the concentrated viral vector that is packaged with the genetic material of interest around the cells, wherein the incubating is effective to transfer genetic material from the viral vector to a subpopulation of the mammalian cell population to form a transfected subpopulation of mammalian cells;
d. selectively labeling the transfected subpopulation of mammalian cells by
(i) incubating the mammalian cell population with a capture particle comprising an agent that recognizes and binds specifically a cell antigen expressed selectively by the transfected subpopulation within the heterogeneous cell population;
(ii) binding the capture particle comprising the agent to the targeted population of cells, to form a labeled transfected subpopulation of cells;
e. passing a wash buffer through the chamber embedded in the centrifuge rotor while the rotor is in motion and the counterflow produces an opposing force within the chamber, wherein the wash buffer removes unbound cells and unbound capture particles from the chamber;
f. collecting in an output bag the transfected subpopulation of cells bound to the capture particle comprising the agent that recognizes the specific cell surface marker so that the cells bound to the agent that recognizes the specific cell surface marker are enriched relative to the heterogeneous cell suspension; and
g. dissociating the cells in (f) from the agent that recognizes the specific cell surface marker,
wherein the method is effective to:
(i) reduce the risk of contamination of the collected cells;
(ii) reduce damage to the collected cells;
(iii) maintain viability of the collected cells; or
(iv) a combination thereof.

57. The method according to claim 56, wherein binding of the capture particle comprising the agent that recognizes and binds specifically to the transfected subpopulation of cells within the heterogeneous cell population is effective to change at least one of size, density and buoyancy of each transfected cell compared to an unlabeled cell in the heterogeneous cell population.

58. The method according to claim 56, further comprising
adding a lysing agent to the chamber embedded in the centrifuge rotor while the rotor is in motion and the counterflow produces an opposing force within the chamber, wherein the lysing agent lyses the bead; and
passing a wash buffer through the chamber embedded in the centrifuge rotor while the rotor is in motion and the counterflow produces an opposing force within the chamber, wherein the wash buffer removes the lysing agent and the lysed bead.
